SEQUENCE LISTING (29 sequences)

SEQ ID NO: 1 - DNA, 596 bp, Aspergillus fumigatus
  modified_base (14)..(101): N = A, C, G OR T/U
tccgtaggtgaacntgcggaaggatcattacngagtgagggccttntgggtccaacttcccaccngtgtctatcgtaccttgttgcttcggcgggcccgcngtttcgacggccgccggggaggcttgcgcccccgggcccgcgcccgccgaagaccccaacatgaacgctgttctgaaagtatgcagtctgagttgattatcgtaatcagttaaaactttcaacaacggatctcttggttccggcatcgatgaagaacgcagcgaaatgcgataagtaatgtgaattgcagaattcagtgaatcatcgagtctttgaacgcacattgcgccccctggtattccggggggcatgcctgtccgagcgtcattgctgccctcaagcacggcttgtgtgttgggcccccgtccccctctcccgggggacgggcccgaaaggcagcggcggcaccgcgtccggtcctcgagcgtatggggctttgtcacctgctctgtaggcccggccggcgccagccgacacccaactttatttttctaaggttgacctcggatcaggtagggatacccgctgaacttaagcatatcaataagcggagga

SEQ ID NO: 2 - DNA, 16 bp, Artificial Sequence
  Description of Artificial Sequence  Synthetic Primer
ttggttccggcatcga

SEQ ID NO: 3 - DNA, 17 bp, Artificial Sequence
  Description of Artificial Sequence  Synthetic Primer
gcagcaatgacgctcgg

SEQ ID NO: 4 - DNA, 25 bp, Artificial Sequence
  Description of Artificial Sequence  Synthetic Primer
tgaagaacgcagcgaaatgcgataa

SEQ ID NO: 5 - DNA, 552 bp, Fusarium sp.
tctccgttggtgaaccagcggagggatcattaccgagttatacaactcccaaacccctgtgaacttacctataacgttgcttcggcgggaaaccgacggccccgtaagacgggccgcccccgccagaggaccccctaactcttgttatatcctgtttctctgagtacaacaagcaaataaattaaaactttcaacaacggatctcttggctctggcatcgatgaagaacgcagcgaaacgcgataagtaatgtgaattgcagaatttagtgaatcatcgaatctttgaacgcacattgcgcccgccagtattctggcgggcatgcctgtccgagcgtcattacaaccctcaggcccccgggcctggtgttggggatcggtcggagccctccggggcacacgccgtcccccaaattcagtggcggtctcgctgcagcctccattgcgtagtagctaacacctcgcaactggaaggcggcgcggccacgccgtaaaacccccccaacttctgaatgttgacctcggatcaggtaggaatacccgctgaacttaa

SEQ ID NO: 6 - DNA, 495 bp, Scedosporium Prolificans
ccgagttattactccaaaccctttgtgaaccttacctatgttctgttgcctcggcggggaggaagaccccttaaaaagggcccagcccccgccggcagcaccaaactcttgcatttatagcggattacatttctgaatacaatacaaaacaaatcaaaactttcaacaacggatctcttggttctggcatcgatgaagaacgcagcgaaatgcgataagtaatgtgaattgcagaattcagtgaatcatcgaatctttgaacgcacattgcgccggcagtaatctgccggcatgcctgtccgagcgtcatttcaaccctcgagcctaggtttttacaagcccaaggatcggtgttggggcgctacggttgtcatctgaccgccgtaggctctgaaatacagtggcggtcccgccgcggcgccttctgcgtagtagatttacaactcgcattgggtcccggcgaaggccagccgtcaaaccctctattcttatggt

SEQ ID NO: 7 - DNA, 19 bp, Artificial Sequence
  Description of Artificial Sequence  Synthetic Primer
cagccgacacccaacttta

SEQ ID NO: 8 - DNA, 22 bp, Artificial Sequence
  Description of Artificial Sequence  Synthetic Primer
tcctccgcttattgatatgctt

SEQ ID NO: 9 - DNA, 19 bp, Artificial Sequence
  Description of Artificial Sequence  Synthetic Primer
tcaagcacggcttgtgtgt

SEQ ID NO: 10 - DNA, 20 bp, Artificial Sequence
  Description of Artificial Sequence  Synthetic Primer
caacggatctcttggttccg

SEQ ID NO: 11 - DNA, 22 bp, Artificial Sequence
  Description of Artificial Sequence  Synthetic Primer
ggatcaggtagggatacccgct

SEQ ID NO: 12 - DNA, 23 bp, Artificial Sequence
  Description of Artificial Sequence  Synthetic Primer
ttgcagaattcagtgaatcatcg

SEQ ID NO: 13 - DNA, 21 bp, Artificial Sequence
  Description of Artificial Sequence  Synthetic Primer
tcaacaacggatctcttggtt

SEQ ID NO: 14 - DNA, 21 bp, Artificial Sequence
  Description of Artificial Sequence  Synthetic Primer
ctgccgacgttttccaaccat

SEQ ID NO: 15 - DNA, 21 bp, Artificial Sequence
  Description of Artificial Sequence  Synthetic Primer
ttgccgaacgcaaatcaatct

SEQ ID NO: 16 - DNA, 21 bp, Artificial Sequence
  Description of Artificial Sequence  Synthetic Primer
cagccgacacccaactttatt

SEQ ID NO: 17 - DNA, 21 bp, Artificial Sequence
  Description of Artificial Sequence  Synthetic Primer
ccgccgacgcatttatttgca

SEQ ID NO: 18 - DNA, 28 bp, Artificial Sequence
  Description of Artificial Sequence  Synthetic Primer
ttctaaggttgacctcggatcaggtagg

SEQ ID NO: 19 - DNA, 31 bp, Artificial Sequence
  Description of Artificial Sequence  Synthetic Primer
cagccgacacccaactttatttttctaaggt

SEQ ID NO: 20 - DNA, 25 bp, Artificial Sequence
  Description of Artificial Sequence  Synthetic Primer
ctgccgacgttttccaaccattctt

SEQ ID NO: 21 - DNA, 26 bp, Artificial Sequence
  Description of Artificial Sequence  Synthetic Primer
ttgccgaacgcaaatcaatctttttc

SEQ ID NO: 22 - DNA, 17 bp, Artificial Sequence
  Description of Artificial Sequence  Synthetic Primer
agaacgcagcgaaatgc

SEQ ID NO: 23 - DNA, 15 bp, Artificial Sequence
  Description of Artificial Sequence  Synthetic Primer
acgcagcgaaatgcg

SEQ ID NO: 24 - DNA, 18 bp, Artificial Sequence
  Description of Artificial Sequence  Synthetic Primer
acggatctcttggttccg

SEQ ID NO: 25 - DNA, 18 bp, Artificial Sequence
  Description of Artificial Sequence  Synthetic Primer
tcaagcacggcttgtgtg

SEQ ID NO: 26 - DNA, 20 bp, Artificial Sequence
  Description of Artificial Sequence  Synthetic Primer
agcgggtatccctacctgat

SEQ ID NO: 27 - DNA, 22 bp, Artificial Sequence
  Description of Artificial Sequence  Synthetic Primer
cgatgattcactgaattctgca

SEQ ID NO: 28 - DNA, 17 bp, Artificial Sequence
  Description of Artificial Sequence  Synthetic Primer
aatgacgctcggacagg

SEQ ID NO: 29 - DNA, 19 bp, Artificial Sequence
  Description of Artificial Sequence  Synthetic Primer
tcagcgggtatccctacct

(19) United States
(12) Patent Application Publication
(10) Pub. No.: US 2005/0009051 A1
(43) Pub. Date: Jan. 13, 2005

(54) Diagnosis of mould infection

(75) Inventors: Xiang-Yang Han, Bellaire, TX (US); Jeffrey J. Tarrand, Houston, TX (US); Audrey S. Pham, Houston, TX (US); Gregory S. May, Houston, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas

(21) Appl. No.: 10/829,661

(22) Filed: Apr. 22, 2004

Related U.S. Application Data
(63) Continuation-in-part of application No. 10/672,300, filed on Sep. 26, 2003.
(60) Provisional application No. 60/414,008, filed on Sep. 27, 2002.

(51) Int. Cl. C12Q 1/68

(52) U.S. Cl. 435/6

(57) ABSTRACT

The present invention provides methods for detecting the presence of pathogenic moulds in biological samples that are based on amplification of mould nucleic acids. The methods may further comprise quantitating and real time detection of the mould. The methods of the invention are highly specific and do not co-amplify human or other yeast nucleic acids. The methods of the invention are also extremely sensitive. Thus, methods for diagnosing infections caused by mould are provided. The invention also provides kits for detection of moulds.

The present application is a continuation-in-part application which claims the benefit of the filing date of U.S. patent application Ser. No. 10/672,300 filed on Sep. 26, 2003 which claims priority to co-pending U.S. Provisional Application, Ser. No. 60/414,008 filed Sep. 27, 2002. The entire text of the above-referenced disclosure is specifically incorporated herein by reference without disclaimer.

### BACKGROUND OF THE INVENTION

1. Field of the Invention

The present invention relates generally to the fields of microbiology and pathology. More particularly, it concerns the development of methods to diagnose mould infections such as invasive mould infections, using real-time PCR™ based methods.

2. Description of Related Art

Aspergillus and other septate moulds are ubiquitous and may cause invasive aspergillosis (IA) or invasive mould infection (IMI) among patients with neutropenia and immunosuppression. These infections carry a fatality rate of 92% (Paterson and Singh, 1999). Patients undergoing hematopoietic stem cell transplantation are particularly vulnerable to this infection with an estimated incidence of 6.4% by patient (Paterson and Singh, 1999). Although nationwide incidence of the disease is unknown, the number of cases has been increasing over the past decade largely due to an increase of the susceptible populations (Latge, 1999).

Thus, definitive and early diagnosis of IMI is crucial for proper patient management, clinical research, and epidemiological studies. A number of factors, however, hamper this effort. First, definitive diagnosis entails tissue sampling by invasive procedures, which is frequently impractical due to associated risks, particularly thrombocytopenia. The diagnosis is often rendered late in the disease course. In fact, many such cases are established by autopsy (Vogeser et al., 1997; Kontoyiannis et al., 2000). Second, immunocompromized patients may be unable to mount an effective immune response, which precludes an antibody-based diagnosis (Latge, 1999). In addition, it is difficult to distinguish between active disease, colonization, or contamination in these patients. Third, many moulds, particularly Aspergillus, are rarely isolated from blood cultures (Tarrand et al., 2000), unlike bacteria. Fourth, isolation from solid tissue is also infrequent for Aspergillus (Tarrand et al., 2000). On the other hand, isolation of moulds from a normal host's airway is not uncommon due to the ubiquitous nature of moulds.

Another facet of the difficulties in diagnosing mould infections is that the infection can be manifested in a broad spectrum of disease in the human host. Mould infection may be categorized according to the severity of the infection (Soubani et al., 2002). For example, aspergillus infection may be categorized as invasive aspergillosis, aspergilloma, chronic necrotizing aspergillosis, or allergic bronchopulmonary aspergillosis. Allergic bronchopulmonary aspergillosis is generally encountered in asthamatic patients and is characterized by both type-I and type-III immune reactions to Aspergillus. These immune responses lead to a marked inflammatory reaction and may result in damaged bronchial walls. Aspergillomas is generally characterized by a tangled growth of Aspergillus hyphae admixed with mucous and cellular debris in a cavity. Aspergillomas is commonly referred to as a fungal ball. Aspergillomas have been most frequently documented as occurring in residual tuberculous cavities. Chronic necrotizing pulmonary aspergillosis is usually found in patients with some degree of immunosuppresion, most commonly associated with underlying lung disease, alcoholism, or chronic corticosteroid therapy. An aspergillus infection that affects the lungs may show up as invasive pulmonary aspergillosis, a presence of colonization, aspergilloma, chronic necrotizing aspergillosis, or allergic bronchopulmonary aspergillosis.

Another difficulty in diagnosing mould infection is distinguishing the invasiveness status from other types of infections. Because infection at the invasive stage may be life-threatening, it is especially urgent that invasive mould infection be diagnosis. Invasive mould infections are an increasing complication of cancers and of their treatment. It is often difficult to determine whether aspergillosis has reach invasive status because of the techniques used in the diagnosis. For example, chest radiograph is a standard diagnostic method to determine whether a patient has invasive aspergillosis. This method may be an adequate diagnostic tool provided the chest is involved in the line of infection. Up to 25% of patient with invasive aspergillosis exhibit normal chest radiograph, indicating infection in tissue sites other than the chest.

Yet another difficulty in diagnosing fungal infection is that current diagnostic methods, which appear promising, have high error rate, are not sensitive, and are non-specific or impractical. Methodologies that have been considered diagnostic standards have been attempted as detection methods for mould infection. However, these methods have yet to resolve the difficulties encountered. For example, antigen assays do not provide sufficient sensitivity or specificity for routine use, histology is too invasive, the sensitivity in culture is low, and antibody detection is suboptimal in neutropenic patients (Eijavec et al., 2002). Most current assays need confirmation by clinical signs or other assays because no assay is sufficient to diagnose mould infection. The inability to obtain conclusive diagnoses has lead to variability in the definitions used by the clinicians and to the start of empirical therapy.

A promising diagnostic approach using PCR™ for the detection of circulating Aspergillus DNA in the bloodstream has emerged in recent years (Bretagne et al., 1998; Einsele et al., 1997; Hebart et al., 2000; Van Burik et al., 1998; Williamson et al., 2000; Yamakami et al., 1996). These studies have shown that PCR™ has the advantages of higher sensitivity, less environmental contamination, and easier and repeatable sampling of blood. However, these studies are all limited by smaller patient populations, possible subjectivity, cross-reactivity to Candida, lack of quantitation, or a combination of all these factors. Thus, the art still lacks a reliable method for detecting and diagnosing mould infections such as invasive mould infections (IMI).

In addition, conventional PCR™ methods, such as one-step amplification followed by detection using agarose resolution gel electrophoresis, are inadequate to detect the presence of exogenous mould nucleic acid. In one aspect, conventional PCR™ assays for the detection of the DNA load are generally too insensitive to provide a reliable screening test. Moreover, conventional PCR™ methods were not designed to be high-throughput systems and are susceptible to PCR™ cross-contamination. In another aspect, a biological sample is often filled with the host's nucleic acid and other potential contaminants. Simplicity and rapidity of methodologies have been compromised in attempts to manipulate sensitivity and specificity. The sensitivity and specificity of each method is significantly affected by reaction conditions. Some methods such as nested PCR™ may even be rendered unfit for clinical diagnosis of mould infection (Ferns et al., 2002). Nested PCR™ is referred to as a layering of PCR™ by use of multiple amplification runs and/or primers. In some nested PCR™, a first run amplifies a DNA fragment of a certain size. A second PCR™ uses the product of the first run as a template and a primer set that amplifies a smaller DNA fragment. The use of nested PCR™ reduces the simplicity and rapidity of diagnosis because the technique requires multiple rounds of PCR™ and additional primer sets to amplify low copy number of nucleic acid templates. Nested PCR™ may be unfit for clinical diagnosis because any nucleic acid contamination may be amplified at least twice, increasing the significance of the contaminant relative to the targeted PCR™ product. Specificity of DNA target may be compromised.

Yet another drawback of current approaches for diagnosing mould infections is that some protocols require days or weeks for diagnosis. For example, detection by culture method averages approximately 3 weeks. By the time a positive result is confirmed, the infection may advance to a fatal stage. It is desirable in the art to have a method requiring a shorter turnaround time. A PCR™ based technique show the most promise in reducing turnaround time. A complete diagnosis may be performed in a matter of hours. PCR™ is the most frequently used amplification procedure because it can readily adapt to many applications. However, current protocols require special amplification steps, such as nested PCR™, to achieve the desired sensitivity. In addition, the product generated is usually analyzed by ethidium bromide-stained gel electrophoresis. Although gel electrophoresis is simple to use, it is much less sensitive than other methods of detection, such as Southern blotting. In addition, detection by ethidium bromide staining and Southern blotting is time-consuming and the interpretation of the results may be subjective. An attempt to solve this problem is by coupling amplification with hybridization of a complementary probe, and the detection of a reaction product that provides either a colorimetric or fluorescence readout.

Therefore, a less invasive, reliable, sensitive, and specific diagnostic test for mould infection such as IMI is urgently needed in the art.

### SUMMARY OF THE INVENTION

The present invention overcomes the deficiencies in the art by providing a less invasive, more reliable, sensitive, and specific diagnostic test for mould infection such as IMI. The present inventors have developed detection methods based on the amplification of mould DNA using primers that specifically and selectively amplify 5.8S ribosomal RNA of a mould. In some embodiments, real-time PCR-based methods are described that combine amplification and simultaneous probe hybridization to achieve sensitive, specific, and quantitative detection of infectious moulds in real time thereby providing instant detection of moulds.

The present invention therefore provides methods for detecting the presence of a mould infection in a subject comprising identifying 5.8S ribosomal RNA of a mould or a DNA encoding the ribosomal RNA, in a sample obtained from the subject. In other embodiments, the present invention comprises mixing the sample with a nucleic acid encoding the 5.8S ribosomal RNA of a mould; amplifying the sample encoding the RNA; and determining the presence or absence of an amplification product in the sample obtained wherein the presence of an amplification product is indicative of the presence of a mould infection. In particular embodiments, the primers hybridize to the 5.8S ribosomal RNA or DNA encoding the RNA under high stringency conditions as disclosed herein. In some embodiments of the present invention, the mould infection may be an invasive mould infection or a non-invasive mould infection.

In further embodiments, the methods of the present invention further comprise quantitating the amplification product whereby the amount of a mould nucleic acid is quantitated. In some aspects the quantitating comprises (a) mixing a first probe capable of hybridizing to a nucleic acid sequence of the mould in an amplification reaction; (b) mixing a second probe capable of hybridizing to a standard nucleic acid that is amplified to a pre-determined quantity in the same amplification reaction; and (c) comparing or quantifying the signal of the nucleic acid in the amplification reaction of step (a) with that of a signal of a amplification reaction containing a standard nucleic acid. The comparing step is in the exponential phase of the amplification reaction. The mixing of the first and second probes occur during the exponential phase of the amplification.

In some specific embodiments, the first or second probe may comprise nucleic acids that hybridize to the nucleic acid sequence of SEQ ID NO:1, SEQ ID NO:5 or SEQ ID NO:6 or fragments thereof. In other specific embodiments, the first or second probe may comprise the nucleic acid sequence 5′-TGAAGAACGCAGCGAAATGCGATAA-3′ (SEQ ID NO:4). In further embodiments, the probe may comprise of SEQ ID NO:14, SEQ ID NO:15, SEQ ID NO:16, SEQ ID NO:17, SEQ ID NO:18, SEQ ID NO:19, SEQ ID NO:20, or SEQ ID NO:21.

In some aspects of the invention, it is contemplated that any of the probes used in the methods may be labeled for detection purposes. Several types of labels are known in the art including fluorescent labels, radioisotopes, colorimetric labels, ligands, antibodies, enzymatic tags and the like. In some specific embodiments, the use of fluorescent labels is particularly contemplated. Some non-limiting examples of fluorescent labels include 6-carboxyfluorescein (FAM), 6-carboxy-N,N,N′,N′-tetramethylrhodamine (TAMRA), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Texas Red, VIC, or DABCYCL. In specific aspects, the probe comprises the sequence 5′-6-FAM-TGAAGAACGCAGCGAAATGCGATAA-TAMRA-3′ (SEQ ID NO:4).

It is contemplated that the nucleic acid from the sample may be RNA or DNA. In embodiments where the nucleic acid is RNA, the amplification reaction is preceded by a reverse transcription reaction.

The moulds that are detected by the methods of the invention include those of the Aspergillus species, the Fusarium species, and/or the Scedosporium species. Some non-limiting examples of moulds of the Aspergillus species include Aspergillus fumigatus, Aspergillus flavus, Aspergillus terreus, Aspergillus vesicularis, Aspergillus nidulans, or Aspergillus niger. Non-limiting examples of moulds of the Fusarium species include Fusarium solani and those of the Scedosporium species include Scedosporium prolificans.

In particular embodiments, the present invention contemplates the use of samples such as biological samples and/or nucleic acid containing samples. Samples are described in detail in the specification and may comprise serum, blood, plasma, cells, tissues, aspirates, biopsies, fine needle aspirates, skin biopsies, lymph fluid or urine.

In some embodiments, primers of the invention are comprised of nucleic acids that hybridize to the nucleic acid sequence comprised in SEQ ID NO: 1 or fragments or variants thereof. In some specific aspects of this embodiment, the primers comprise the nucleic acid sequence TTGGTTCCGGCATCGA (SEQ ID NO:2) or GCAGCAATGACGCTCGG (SEQ ID NO:3). In other embodiments of the invention, the primers comprise the nucleic acid sequence SEQ ID NO:9, SEQ ID NO:10, SEQ ID NO:13, SEQ ID NO:22, SEQ ID NO:23, SEQ ID NO:24, or SEQ ID NO:25. In other embodiments, primers comprise the nucleic acid sequence SEQ ID NO:11, SEQ ID NO:12, SEQ ID NO:26, SEQ ID NO:27, SEQ ID NO:28, or SEQ ID NO:29.

In still other embodiments, the primers are comprised of nucleic acids that hybridize to the nucleic acid sequence comprised in SEQ ID NO: 5 or fragments or variants thereof. In yet other embodiments, the primers are comprised of nucleic acids that hybridize to the nucleic acid sequence comprised in SEQ ID NO: 6 or fragments or variants thereof. In still yet other embodiments, the primers are comprised of nucleic acids that hybridize to the nucleic acid sequence comprised in SEQ ID NO:7, or SEQ ID NO:8 or fragments or variants thereof.

The amplification may be carried out by polymerase chain reaction (PCR™). However, other forms of amplification known in the art may be used as well. A brief description of PCR™ and certain other amplification reactions is provided infra in the specification.

In some embodiments of the methods, identifying or determining the presence of invasive mould is in real time. The methods of the invention are extremely sensitive with a detection range greater than 0. In particular embodiments, the detection range may be 1 fg to 20 ng, or 1 fg to 800 fg, or more preferrably 100 fg to 200 fg of DNA. The methods of the invention are highly specific and do not amplify or detect human or candidal DNA.

In some embodiments, the methods further comprise obtaining a sample such as a biological or nucleic acid containing sample from a subject. In other embodiments, the methods further comprise isolating nucleic acids from the biological sample.

In another particular embodiment of the present invention, there is provided a method for purification of a nucleic acid encoding 5.8S ribosomal RNA of a mould in a nucleic acid containing sample comprising (a) obtaining the nucleic acid containing sample from a subject; (b) incubating the sample with a lysis reagent for about at least about 60 minutes; (c) vortexing the sample intermittently to mix; and (d) centrifuging the sample at greater than 3000×g for 5 min.

The sample may be incubated at about 36° C., about 36.5° C. about 37° C., about 37.5° C., about 38° C., about 38.5° C., about 39.5° C., about 40° C., about 40.5° C., about 41° C., about 41.5° C., about 42° C., about 42.5° C., about 43° C., about 43.5° C., about 44° C., about 44.5° C., about 45° C., about 45.5° C., about 46° C., about 46.5° C., about 47° C., about 48° C., about 48.5° C., about 49° C., about 49.5° C., about 50° C., about 50.5° C. or greater. In a preferred embodiment, the sample may be incubated at about 37° C. or at about 50° C. In is also contemplated in the present invention, that the sample may be centrifuged at about 6000×g for 15 minutes.

In yet another particular embodiment of the present invention, there is provided a method for enhancing binding of a nucleic acid encoding 5.8S ribosomal RNA of a mould to silica beads comprising (a) washing the silica beads with sodium acetate of about approximately pH 5.2; (b) mixing the silica beads of step (a) by vortexing; and (c) centrifuiging the silica beads at about 1000 rpm for at least about 1 minute. The silica beads of step (a) may be washed at least 2, at least 3, at least 4, at least 5 or more times. In a preferred embodiment, the silica beads are centrifuged at least at about 12000 rpm. The method of enhancing binding of a nucleic acid encoding 5.8S ribosomal RNA of a mould to silica beads comprises selecting silica beads of particle size ranging from 5 μM to 10 μM.

The method for enhancing binding of a nucleic acid encoding 5.8S ribosomal RNA of a mould to silica beads comprises washing the silica beads with sodium acetate of about 0.05 M to 2.5 M. In some embodiments of the invention, washing the silica beads with sodium acetate of 0.1 M is preferred.

In still yet another embodiment of the invention, the method for enhancing binding of a nucleic acid encoding 5.8S ribosomal RNA of a mould to silica beads further comprises mixing the silica beads with a nucleic acid containing sample from a subject comprising a nucleic acid encoding 5.8S ribosomal RNA of a mould.

The invention also provides kits for detecting a mould in a biological sample. Such kits comprise (a) primers that hybridize to the 5.8S ribosomal RNA of a mould or a DNA encoding the RNA; and b) reagents for an amplification reaction comprising a heat-stable DNA polymerase enzyme, buffers, water, magnesium chloride, and deoxynucleotides; each enclosed in a suitable container means.

In some embodiments, the kit may comprise primers comprising nucleic acids that hybridize to the nucleic acid sequence of SEQ ID NO:1, SEQ ID NO:5, SEQ ID NO:6, SEQ ID NO:7, or SEQ ID NO:8 or fragments thereof. In other embodiments, primers of the kit comprise the nucleic acid sequence 5′-TTGGTTCCGGCATCGA-3′ (SEQ ID. NO:2) or 5′-GCAGCAATGACGCTCGG-3′ (SEQ ID NO:3). In further embodiments, the kit may comprise primers comprising the nucleic acid sequence of SEQ ID NO:9, SEQ ID NO:10, SEQ ID NO:13, SEQ ID NO:22, SEQ ID. NO:23, SEQ ID NO:24, or SEQ ID NO:25. In still a further embodiment, the kit may comprise a primer comprising the nucleic acid sequence SEQ ID NO:11, SEQ ID NO:12, SEQ ID NO:26, SEQ ID NO:27, SEQ ID NO:28, or SEQ ID NO:29.

In other embodiments, the kits may further comprising one or more probes that hybridize to the 5.8S ribosomal RNA of the mould or fragments thereof, or a DNA encoding the RNA. It is contemplated that these probes will comprise a nucleic acid sequence that hybridizes to the nucleic acid sequence of SEQ ID NO:1, SEQ ID NO:5 or SEQ ID NO:6 or fragments thereof. In specific aspects, the kit will comprise one or more probes comprising the sequence 5′-TGAAGAACGCAGCGAAATGCGATAA-3′ (SEQ ID NO:4). In further embodiments, the kit may comprise one or more probes of SEQ ID NO:14, SEQ ID NO:15, SEQ ID NO:16, SEQ ID NO:17, SEQ ID NO:18, SEQ ID NO:19, SEQ ID NO:20, or SEQ ID NO:21.

In yet other aspects, the one or more probe is labeled with one or more suitable detectable label(s). Alternatively, labels may be provided with instructions on how to label the probe(s) with the label.

The kits of the invention may also comprise reagents to isolate nucleic acids from a sample, such as a biological sample and/or a nucleic acid containing sample. In some embodiments, reagents used to isolate mRNA will be comprised in such a kit. In other embodiments, reagents used to isolate DNA will be comprised in such a kit.

It is also contemplated that the kits will comprise suitable standards that may be amplified and/or detected and/or quantified. This will include both negative and positive standards as known in the art.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. Detection of mould (Aspergillus) DNA by real-time PCR. FIG. 1A. Detection signal (ΔRₙ) and PCR™ cycles versus the quantity of Aspergillus DNA: (●) 0 fg, (∘) 2×10² fg, (▪) 2×10³ fg, (□) 2×10⁴ fg; (▴) 2×10⁵ fg, (∇)2×10⁶ fg, and (X) 2×10⁷ fg. Horizontal line represents the threshold of detection cycle. FIG. 1B. A semi-log standard curve of FIG. 1A. from another experiment. C_{T}=detection cycles. (●)=Aspergillus DNA standard. (∘)=serum being tested.

FIG. 2. Quantities of mould DNA in 559 sera from patients with “documented”, “probable”, “possible”, and “unlikely” IMI tested by real-time PCR™. Signals below the negative control (equivalent to 10 fg) were given an arbitrary quantity between 1 to 10 fg for the purpose of plotting and logarithmic conversion.

### DESCRIPTION OF ILLUSTRATIVE EMBODIMENTS

A. The Present Invention

Invasive mould infections (IMI) that infect predominantly immunocompromised patients have poor outcome and present a diagnostic challenge. For example, the detection of Aspergillus infection has been difficult as blood, urine or cerebrospinal fluid cultures are rarely positive, although, the fungi can be seen in smears and biopsies from infected tissue. Thus, typical diagnostic methods more than often require invasive or surgical procedures that would cause additional detrimental effects to the already immunocompromised patient.

The present inventors have developed diagnostic assays based on PCR™ to diagnose mould infection such as IMI by detecting mould nucleic acids in the serum. To achieve the goals of simple, early and rapid detection of mould infections, a real-time quantitative PCR™ method was adopted. Real-time PCR™ is referred to as the ability to monitor the progress of PCR™ as it occurs (i.e., in real time). The advantage over conventional PCR™ is that detection of the PCR™ product is integrated with the amplification phase, thus negating a need for any identification, detection or post-PCR™ processing. Furthermore, the processing time for diagnosis is greatly reduced without the need for identification, detection or post-PCR™ processing steps.

The technique of the present invention offers other significant advantages over current techniques used in the art to identify mould infection. For example, in the present invention, the use of multiple primer sets is not require. Rather, the present invention employs well-defined primers to specific regions of the nucleic acid sequence(s) for PCR™. A further advantage of the present invention is that PCR™ is conducted at relatively high stringency, thereby decreasing contamination of the host DNA. Another advantage of the present invention lies in the use of non-specific primers and the low annealing temperature in PCR™ which increases the reproducibility. Further, species identification may be performed by hybridizing a species-specific probe to the nucleic acids sequence of a PCR™ product. The technique of the present invention is further advantageous over current techniques in that the assay does not required the use of complex amplification methods, such as nested, or semi-nested PCR™ methods. However, in some embodiments of the present invention, such amplification methods may be employed. In some embodiments, use of multiplex or multicomponent PCR™ may be preferred. Further, the assay obviates the need to conduct additional biochemical tests, which reduces time, labor and expense.

The present invention utilizes the nucleic acid sequence of the 5.8S ribosomal RNA of Aspergillus. The 5.8S rDNA contains intraspecies and interspecies DNA sequence variations among species of Aspergillus. Prior techniques, have utilized other nucleic acid regions, have steered clear of exploiting these variations so as to obviate the need for necessary extensive sequencing and characterization of resultant PCR™ products in order to identify isolates, at least at the species level. In contrast, the present invention utilizes genus-specific PCRs which specifically target variation in sequences both within and between species of Aspergillus. Species determination offers a second tier of discrimination for clinicians to tailor treatments. For example, antifungal trimethyltinbenzoate-4-picoline was found to be more effective against A. niger than A. fumigatus (Choudhury et al., 2001).

Thus, the present invention provides methods to detect and diagnose pathogenic moulds that cause IMI including those of the Aspergillus species, Fusarium species and Scedosporium species in biological samples such as serum, plasma, blood, or other body fluids that can be easily obtained and therefore provide a significant advantage in the early detection and treatment regimen of such infections. Assays of the present invention are extremely sensitive and can detect 0 fg or greater, preferably 10 fg to 20 ng (6-log range) mould DNA and most preferably 110 fg which is estimated to be equivalent to that of 0.3 to 30 genomes in the case of moulds of the Aspergillus species, Fusarium species and Scedosporium species. The methods are also highly specific in selectively amplifying and identifying the mould DNA as evidenced by the failure to co-amplify or detect human or candidal DNA.

Thus, the novel features of the present allow for a rapid, high resolution, quantitative determination of large number of samples for any mould infecting species such as Aspergillus.

B. Invasive Moulds and Infections

Infections by invasive fungal pathogens are responsible for the mortality of patients that are immunosuppressed including, patients afflicted with hematological cancers, post-operative patients, transplant patients, those infected by HIV, those undergoing chemotherapy, and cancer patients receiving immunosuppressive medications. As discussed in a recent review by Brakhage and Langfelder (2002), species of the Aspergillus family account for majority of these fungal infections. Invasive aspergillosis alone is associated with a high mortality rate that ranges from 30% 1to 90% (Brakhage and Langfelder, 2002). Moulds of the Fusarium species and Scedosporium species are also responsible for severe infections that are associated with mortality in immunosuppressed patients (Oliveria et al., 2002; Baddley et al., 2001).

Aspergillosis is the most common mould infection in immunosuppressed patients. Aspergillosis is a term that encompasses a variety of disease processes caused by Aspergillus species. Aspergillus species are ubiquitous; their spores are constantly being inhaled. Of the more than 300 species known, some are opportunistic pathogens for man and these include: A. fumigatus, A. flavus, A. niger, A. nidulans, A. terreus, A. sydowi, A. flavatus, A. glaucus, and A. vesicularis. Aspergillosis is increasing in prevalence and is particularly a problem among patients with chronic respiratory disease or immunocompromised patients. Opportunistic pulmonary aspergillosis is characterized by widespread bronchial erosion and ulceration, followed by invasion of the pulmonary vessels, with thrombosis, embolization and infarction. Clinically, infection manifests as a necrotizing patchy bronchopneumonia, sometimes with hemorrhagic pulmonary infarction. In about 40% of cases, there is hematogenous spread to other sites. Aspergillosis is also a rare but devastating complication of traumatic wounds, such as, bum wounds, frost bite wounds, or wounds developed by diabetics, where amputation is often required for cure. Invasive aspergillosis is commonly fatal, so aggressive diagnosis and treatment is required.

C. Case Definition for IMI

Cases of IMI are defined herein according to the criteria established by EORTC and the Mycoses Study Group (Ascioglu et al., 2002). Depending on the degree of diagnostic certainty, the cases are defined as “definitive or documented”, “probable”, “possible”, and “unlikely” IMI. Consequently, a clinical diagnosis of “probable” or “possible” IMI is frequently necessary, made through a correlation of clinical manifestation, radiological findings, microbiological culture, and exclusion of other etiologies (Ascioglu et al., 2002). “Possible” invasive fungal infection is described as having at least 1 host factor criterion, and 1 microbiological or 1 major (or 2 minor) clinical criteria from abnormal site consistent with infection. “Probable” invasive fungal infections is described as having at least 1 host factor criterion, and 1 microbiological criterion, and 1 major (or 2 minor) clinical criteria from abnormal site consistent with infection. “Proven”. invasive fungal infections are further classified into 3 sub-groups of deep tissue infections, fungemia, or endemic fungal infections. Deep tissue infection of mould occurs when histopathologic or cytopathologic examination show hyphae from needle aspiration or biopsy specimen with evidence of associated tissue damage or when culture obtained by sterile procedure showed positive. Fungemia of moulds are blood culture that yield fungi, excluding Aspergillus species and most species of penicillium. Endemic fungal infections may be either disseminated, systemic or confined to lungs which must be proven by culture.

Briefly, “definitive or documented” IMI represent a tissue diagnosis where branched septate hyphae, inflammation, and necrosis are seen microscopically and/or the fungus is successfully cultured from the tissue. Most of these patients have pulmonary IMI and are typically neutropenic and/or immunosuppressed for an extended period of time and in general exhibit prolonged pneumonia unresponsive to anti-bacterial therapy with nodular and/or cavitory lesions in the lung radiologically. Patients with “probable IMI” are also typically immunocompromised and have clinical and radiological features of IMI, and Aspergillus or other septate moulds are isolated twice or more from respiratory specimens (such as sputa, bronchoalveolar ravages, and bronchial washings). In some cases of “probably IMI” although pneumonia is seen in autopsy, moulds are not observed histologically or cultured microbiologically from the autopsy lung tissue. Such cases are diagnosed as “probable IMI.” Patients with “possible IMI” are those with atypical pulmonary radiology, pneumonia unresponsive to anti-bacterials, and isolation of mould once (rarely without) from the airway. Patients who did not meet the criteria for possible IMI represented “unlikely IMI.”

The term “invasiveness” is generally referred to as a rapidly progressive, often fatal infection that occurs in patients who are severely immunosuppressed. The infection is characterized by invasion of blood vessels and cavitary. Groups of immunosuppressed patients include those who are profoundly neutropenic, those who have received bone marrow or solid organ transplants, patients with advanced AIDS or chronic granulomatous disease. In these patients, Aspergillus may be hematogenously disseminate beyond the lung, potentially causing endophthalmitis, endocarditis, and abscesses in the myocardium, kidney, liver, spleen, soft tissue and bone.

D. Biological Samples and Methods of Obtaining Samples

A “biological sample” or “sample” is defined herein as any cell, cellular extract, tissue, organ or bodily fluid. This includes tissue sections, specimens, aspirates, biopsies including bone marrow aspirates, tissue biopsies, tissue swabs, fine needle aspirates and even skin biopsies. Other suitable examples are fluids, including samples where the body fluid is peripheral blood, serum, lymph fluid, cerebrospinal fluid, seminal fluid or urine. Further examples of samples contemplated in the present invention may include but are not limited to sputum, bronchial alveolar lavage (BAL), bronchial wash, tissue biopsy (e.g., lymph node, lung, endometrial curettings, bone, comeal scrapings), body fluids (e.g., cerebral spinal fluid, pleural, synovial, pericardial fluids, peritoneal), pus, bone marrow aspirate, gastric aspirate/wash, stool, tissue fluid (e.g., from eyebrows, ear lobes), implanted medical device, or prosthetic device; or from an environmental source such as soil, sludge, water or liquids.

Sources of sample collection may play a role in determining whether the type of infection has reached the status of invasive. For example, a sample obtained from serum that is positive for Aspergillus has a high probability that the infection is invasive because the Aspergillus DNA that is present in the serum is mostly due to dissemination of the infection from its origin. The origin of Aspergillus infection is not commonly known to be the bloodstream. Serum may be preferred because the sample is relatively much cleaner than other sources, such as blood, and there is much less interference of host nucleic acid and other contaminants than other sources. However, blood may provide adequate sample source in some embodiments. More importantly, a positive result from serum may indicate that the infection status is of an invasive nature.

The present invention discloses methods comprising, in part, providing or obtaining samples from a human subject. In instances where the invention involves collection of a sample of peripheral blood from a human subject, such a sample may be obtained through intravenous withdrawal of blood or other available means from any exterior limb or other vein comprising part of the peripheral circulatory system.

E. Sample Preparation

Methods of purifying nucleic acids adhere to some fundamental techniques. Generally, cells are ruptured to expose the DNA and then the nucleic acids are separated from the crude mixture. Nucleic acids may be separated from the crude extract by use of physical methods such as centrifugation, pressure techniques or by introduction of a medium with higher affinity for the nucleic acid, such as silica, for specific binding. After sufficient washing, the pure nucleic acids may be isolated and suspended in either water or a buffered solution. Some methods are more labor intensive and time consuming than other methods. For example, a method of nucleic acid purification by phenol-chloroform extraction may require repeated extraction of each step at least two times to ensure a nucleic acid yield of high purity. In some embodiments, a method of rapid purification may involve the combined processes of extraction, separation and suspension of DNA in one solution. Methods of rupturing cells may include the use of a detergent such as sodium dodecyl sulfate or a solvent such as phenol-chloroform. It was commonly assumed that any method of purification may be sufficient for isolating DNA.

It is unappreciated in the art that specimen preparation can have a significant impact on the sensitivity and reproducibility of a molecular diagnostic test. Inadequate sample preparation considerably reduces the efficacy of the diagnostic method by producing poor yield or poor nucleic acid quality. Extraction and purification of an exogenous nucleic acid from clinical samples have proved to be especially challenging because of the presence of host nucleic acid in biological samples. The choice of sample preparation may render many PCR-based applications unsuitable for clinical applications. For example, the host DNA may effectively inhibit, by competition, the target DNA. To provide another example, agents, such as heparian or EDTA, that are be added to facilitate sample preservation are well known in the art as inhibitors of nucleic acid amplifications by PCR. In general, the sample preparation method should (1) release cellular DNA from the fungal cell wall and/or thick capsule; (2) concentrate DNA target that may be present in very small amount; and (3) should eliminate protein debris, contaminants, potential inhibitors, and other extraneous materials, without degrading the target DNA. There are currently many protocols for sample preparation, including many commercial kits such as those from Qiagen™ and Promegam™. However, the prior art lack a method for extracting, purifying and concentrating fungal DNA that is appropriate for all situations. More specifically, the art lacks a method that is appropriate for clinical diagnosis of mould infection.

Methods of isolating an extremely low amount of a nucleic acid of foreign DNA, such as that of Aspergillus, from a sample containing an abundance of a host's nucleic acids, such as that from human, may prove to be challenging. Many nucleic acid isolation techniques do not discriminate between foreign nucleic acids and host's nucleic acids. As a result, there is an abundance of literature documenting the feasibility of their PCR-based techniques toward clinical applications. For example, Raad et al. (2002) reported a technique for diagnosis of invasive pulmonary Aspergillus using PCR-based detection of Aspergillus in BAL. The authors reported isolating DNA from BAL “using a standard protocol”, which includes lysis with sodium dodecyl sulfate and protein kinase followed with phenol/chloroform extraction. The authors' result showed a high correlation between groups with proven Aspergillus infection and samples positive for Aspergillus DNA by their technique. However, primers that were used contained significant stretches of DNA that are complementary to human nucleic acids, in a sufficient manner to produce positive results in samples that lack target nucleic acids. It was not confirmed in this study, whether positive results were in fact that of Aspergillus DNA.

Through extensive experimentation, it was independently discovered that not all nucleic acid isolation methods produce results adequate for most applications. In addition, a report comparing different methods of isolation (Fahle et al., 2000) corroborated with the discoveries of the present invention pertaining to DNA isolation of Aspergillus DNA. One factor affecting the efficacy of an isolation method is the source of nucleic acids. Wherein the source of nucleic acid is abundant, such as obtaining human DNA and amplifying a region of the human DNA, any typical method of isolation will probably perform adequately. Wherein nucleic acids are foreign materials in a host or present in very small amount, techniques to increase production of the nucleic acid materials and/or to have highly efficient isolation techniques in order to produce an adequate amount of nucleic acid for a reliable routine analyses is required. Because it is important to maintain the integrity of sample source and results, it was not feasible to increase production of the nucleic acid materials. Therefore, a highly efficient isolation technique is required to isolate sufficient amount of nucleic acid for analyses.

F. Isolation of DNA from a Sample

Efficient DNA extraction is crucial for any PCR-based or other amplification assay, particularly to detect microbial DNA from a biological sample. One of skill in the art will appreciate that any DNA isolation method known in the art may be used (for example, DNA isolation methods set forth in Maniatis et al. (1988), incorporated herein by reference). The success of using these methods depend upon the quantity of the starting DNA material. The quantities of the desired nucleic acids in clinical samples, and particularly those containing nucleic acids of aspergillus, are not high enough for any indiscriminate purification technique to yield sufficient products suitable for molecular diagnostics. Therefore, the present invention employs a silica-binding method that is capable of extracting extremely low levels (picogram) of fungal DNA along with microgram levels (10⁶-fold more) of human DNA in a serum sample. Experimental details of the method are described in the section entitled as “Example 1.” This method is superior to the typically used phenol-chloroform methods and some commercial DNA extraction kits. A previous study (Fahle et al., 2000), also reported that the silica-binding method was the best among six methods in extracting cytomegalovirus DNA from serum and cerebrospinal fluid.

The inventors prefer serum to extract mould DNA because it appears to be a better source than plasma or whole blood for the extraction of fungal or bacterial DNA (Yamakami, et al., 1996; Boom, et al., 1990; Bowman et al., 2001; Stynen et al., 1995; Fahle et aL, 2000; Zerva et al., 2001). In addition, serum typically has much less human DNA (1-5 g/ml) than whole blood (30-50 g/ml), which might favor the extraction of minute quantities of exogenous microbial DNA. However, the inventors recognize that plasma, whole blood, or other sources defined in Section D may be suitable sample in some embodiments.

G. Isolation and Quantitation of RNA Transcripts from the Sample

Optionally, one may isolate RNA from a biological sample which may then be reverse transcribed to DNA, prior to amplification. Many methods to isolate total cellular RNA are well know to those skilled in the art. See, for example, Chomczynski and Sacchi (1987). A particular method to accomplish this task is the use of the Trizol reagent (Gibco Life Technologies) to extract total cellular RNA. The Trizol procedure involves homogenization of the cells in a blender followed by extraction with the phenol- based Trizol reagent. The RNA is then precipitated with isopropyl alcohol and washed with ethanol before being redissolved in RNAse-free water or 0.5% SDS. The inventors prefer the use of silica-based method for isolation of RNA. Methods of purification disclosed below are also applicable for RNA as well as DNA.

H. Reverse Transcription

Reverse transcription (RT), is a process for the conversion of mRNA into DNA. Briefly, a poly-dT primer is annealed to the poly-A tail of a messenger RNA. This provides a free 3′ end for extension by reverse transcriptase (RT). The enzyme performs 5′→3′ synthesis, using the mRNA as a template. The intermediate product, a hybrid RNA-DNA molecule, is created. At the end of this reaction, the enzyme “loops back” on itself by using the last few bases of the reverse transcript as a template for synthesis of a complete, i.e., a complementary DNA that displaces the mRNA. This creates a “hairpin” structure. The original mRNA can then be degraded by alkali treatment, producing a single-stranded DNA. The hairpin provides a natural primer for the next step—the use of DNA polymerase I to convert the single-stranded DNA into double-stranded DNA, or a cDNA. The hairpin is removed by S1 nuclease.

Methods of reverse transcribing RNA into cDNA are well known and described in Sambrook et al. (2001). Alternative methods for reverse transcription utilize thermostable, RNA-dependent DNA polymerases. These methods are described in WO 90/07641 filed Dec. 21, 1990.

I. Purification Methods

1. Automated Method of Purification

In the present invention, automated methods of purification may be performed to obtain a more uniform and reliable product. Boom et al. (1990) reported a rapid and simple silica-based method for nucleic acid purification, Nuclisensm™ (bioMerieux, Durham, N.C.). The automated method of Nuclisensm™ is a pressure driven, closed system, which reduces the probability of contamination from exogenous origin. However, one of the main disadvantages is that samples containing high amount of Aspergillus nucleic acids or contaminants often encounter pressure problems. Once this problem is encountered, the sample must be discarded because the unique cartridge holding nucleic acids makes it virtually impossible to salvage the nucleic acids. In the present invention some modifications have been made to resolve the pressure issue.

In some embodiments, incubation of the samples at least 2 times longer than the manufacturer's recommended incubation time of 30 minutes, with intermittent vortexing of the sample, followed by a spin at approximately 6000×g or higher. A spin of approximately 12,000×g is preferred. In some cases, a spin of 12,000×g is not practical because the tube containing the sample can not withstand the high speed. In these situations, a spin of 6000×g for 15 minutes is preferred. This modified protocol allows for a more efficient DNA isolation, and at the same time eliminates the pressure problem. The longer incubation time coupled with intermittent vortexing facilitates efficient cell lysis and DNA binding to the silica. In some cases, an overnight incubation may be desirable. A high speed spin will allow for the silica-nucleic acid complex to pack to the bottom of the tube. The supernatant is then decanted and fresh lysis buffer is added, then vortex to mix. The sample is then ready to be loaded onto the Nuclisensm™ extractor.

In some embodiments where samples are extremely dirty, such as those from whole blood, incubation at approximately 50° C. will allow for a more efficient cell lysis. In some embodiments, an incubation at 50° C. for 1 hour, with intermittent vortexing, followed by a spin at approximately 12,000×g is preferred.

2. Manual Method of Purification

The Nuclisens™ method requires the use of a unique disposable plastic cartridge for each sample preparation. However, in some cases it may be undesirable to use the Nuclisensm™ method due to cost constraints, pressure problems or other limiting factors. In the present invention, the isolation method has been adapted to a manual method, to be performed without the use of the automated Nuclisensm™, without compromise to the yield. The isolation method disclosed herein may be considered as an improvement of the Boom method. However, use of the Boom method coupled with conventional PCR may not be suitable for clinical application. Certain modifications need to be made to improve the yield of mould nucleic acids. The method described in the present invention exploits each factor considered crucial for efficient isolation of nucleic acid materials.

A factor that is crucial for efficient isolation of a nucleic acid is the use of appropriately treated silica. Silica are small glass beads that has a high affinity to negatively charged particles, such as nucleic acids. Most purification kits rely on the use of silica (e.g., Qiagen™ kits, Wizard™ kits from Promega, Boom method). The efficacy of purification is highly dependent on whether maximum binding to silica beads can be achieved. To achieve maximum binding, silica must be selected and treated. Silica may be purchased from a manufacturer in a variety of sizes. For example, Silica from Sigma (St. Louis, Mo.) has a particle size between 0.5-10 μm, with approximately 80% between 1-5 μm. When there is a high disparity in the sizes between target nucleic acids and contaminant nucleic acids (e.g., mould DNA versus human genomic DNA), a selection for target nucleic acid must be made to maximize yield. The silica is prepared to preferentially bind to small fragments of nucleic acids, such as those belonging to Aspergillus and other moulds, over larger segments, such as those belonging to human genomes. Preferential binding of nucleic acid greatly enhances purification and yield. A tightly packed silica medium will select for the smaller size nucleic acid (mould DNA) over the larger size contaminant nucleic acids. Most commercial kits have not exploited this factor and hence have limited value for the purification of Aspergillus nucleic acids from clinical samples.

The Boom method offers an improvement above the commercial kits because it first suggested the selection of silica size for maximum binding of target nucleic acids. Uniformity of particle sizes is reported to be achieved through a series of H₂O washes. The solution is then treated with 32% HCl solution and autoclaved.

An improved method of particle size selection and treatment of silica for maximum binding may be achieved by the following method: weigh 0.5 g of silica in the size range of 0.5-10 μm and place in a 1.5 mL epitube; add 1 mL of H₂O; autoclave at 121° C. for 20 minutes; and perform a series of washes with 1.5 mL of 0.05 through 2.5 M sodium acetate (pH 5.2/HCl) (NaAc). Washes with 0.1 M NaAc are preferred. A wash is performed by adding 1.5 mL of NaAc, vortex to thoroughly mix and then spun in a microcentrifuge at approximately 1000 rpm of the lowest setting for 1 minute. Washes may be repeated approximately at least 5 times to select for particle sizes in the 5-10 μm range. After the washes, the silica is spun at high speed (approximately 12,000 rpm or higher), all liquid is emptied and 50-100 μL of 0.1 M NaAc is added to suspend the silica particles. Sodium acetate is normally used in nucleic acid precipitation; however NaAc can be used to maximize binding of nucleic acids to the silica particles. The positive charge on the sodium acts as a counter-ion to the negatively charged group of phosphates on the nucleic acids and reduces the repulsive forces, enhancing binding to the silica. Also, because ethanol depletes the hydration shell from nucleic acids, ethanol precipitation is facilitated if cations are available to neutralize the charge on the exposed phosphate groups. The NaAc serves a dual purpose of facilitating nucleic acid binding to silica and precipitation. In some embodiments, treated silica, as described, is used in isolating mould nucleic acids.

Aliquots of 10-mL of Lysis Buffer may be prepared by dissolving 5 M guanidine thiocyanate (GuSCN) to a 50 mM MOPS (pH 7.2/HCl) solution containing 0.2% Triton X-100 and 15% (v/v) ethanol. Wash buffer may be prepared by dissolving 5 M GuSCN to a 50 mM Tris (pH 7.2/HCl) solution containing 15% (v/v) ethanol.

In some embodiments, nucleic acid isolation is performed by adding 1-3 mL of a sample (e.g., serum) to an aliquot of Lysis Buffer. A sample of 2 mL is preferred. The sample is incubated at 37° C. for 1 hour, vortexing every 15 minutes. 50 μL of silica is added to the sample aliquot 30 minutes into the incubation. The sample is spun at approximately 12,000×g for 5 minutes and the supernatant decanted. In some cases, the composition of the tube precludes a high spin at 12,000×g. A spin at 6,000×g for 15 minutes is recommended. The wash is repeated twice by adding 10 mL of Wash Buffer, vortexing and spinning, followed by two washes with 100% alcohol. The sample can then be dried overnight or washed twice with acetone to remove traces of liquids. Sample is then eluted with 50 μL TE buffer or water.

Elution of the sample may be achieved by adding TE buffer or water to the tube containing the dried silica an pipeted to mix thoroughly. The composition is transferred to a 0.2-0.45 μm micro-spin filter tubes and spun at 12,000 rpm for 2 minutes to release the nucleic acids.

J. Amplification Methodology

1. Primers

In general, nucleic acid amplification methodology relies upon the use of primers, which facilitate the amplification process. The word primer, as defined herein, is meant to encompass any nucleic acid that is capable of priming the synthesis of a nascent nucleic acid in a template-dependent process. Typically, primers are oligonucleotides from ten to twenty-five base pairs in length, but longer sequences can be employed. Primers may be provided in double-stranded or single-stranded form, although the single-stranded form is preferred. Specific embodiments of the present invention disclose primers for use in the amplification reactions. “Oligonucleic acids” as used herein refer to a DNA segment or fragment that is shorter than the length of the genome of such DNA segment or fragment. The terms segment and fragment may be used interchangeably in the present invention. An “oligonucleic acids primer,” a “nucleic acids primer,” a “PCR™ primer,” and a “primer” are equivalent terms as used in the present invention and refer to a nucleic acid fragment or an oligonucleic acids which is substantially complementary to a nucleic acid template sequence and may be adapted to prime the extension of DNA during PCR™. A PCR™ primer of the present invention is capable of specific hybridization, under appropriate conditions of buffer, ionic strength and temperature, to a region of the DNA template. An oligonucleic acids primer may be a DNA fragment of any length sufficient to anneal to a nucleic acid template. In some embodiments, an oligonucleic acid primer contemplated in the present invention may be a DNA segment of about 10-50 bases long. In other embodiments, an oligonucleic acid primer may be a DNA segment of about 20-25 bases long.

Primers are designed according to a set of selection criteria, including having a melting temperature of certain range, and substantial complementary to nucleic acid fragments of particular templates while excluding nucleic acid fragments from potential contaminants. Primers may be selected for complementation to a nucleic acid fragment and located as close to the probe as possible without overlapping it, for amplification of a fragment of approximately 50-600 base pairs, the GC content is approximately between 40-60% range and have melting temperatures of approximately 58° C. -61° C., approximately 8° C. to 10° C. below the melting temperature of the probe. In some embodiments, Primer Express™ software may be used as a starting point to guide in the design of primers.

As used herein the term “genus” is generally refer to a principal rank in the taxonomic hierarchy, falling below the family level and above the species level. “Species” is used to refer to a fundamental rank in the taxonomic hierarchy falling below the genus level and indicating the limit of organisms able to interbreed. A “strain” is used to refer to a taxonomic level below the species level, which may indicate population variation within a species. For purposes of PCR™ and other molecular applications, the designation of genus-specific or species-specific may be based upon similarity, or lack thereof, among nucleic acid sequences across genera or species. Designations based on other factors may also be considered. Accordingly, PCRs referred to as “species-specific” are those that are adapted to amplify designated regions of DNA from a species. Those PCRs referred to as “genus-specific” are adapted to amplify designated regions of DNA from a number of species within a particular genus.

Examples of genus-specific primers (forward 5′-3′ are SEQ ID NOS:2, 9, 10, 13 and SEQ ID NOS:22-25) and (reverse 5′-3′ are SEQ ID NOS:3, 11, 12 and SEQ ID NOS:26-29) which may be appropriate in the present invention are:

| | |
|---|---|
| Forward 5′-3′: | |
| TTGGTTCCGGCATCGA; | (SEQ ID NO:2) |
| TCAAGCACGGCTTGTGTGT; | (SEQ ID NO:9) |
| CAACGGATCTCTTGGTTCCG; | (SEQ ID NO:10) |
| TCAACAACGGATCTCTTGGTT; | (SEQ ID NO:13) |
| AGAACGCAGCGAAATGC; | (SEQ ID NO:22) |
| ACGCAGCGAAATGCG; | (SEQ ID NO:23) |
| ACGGATCTCTTGGTTCCG | (SEQ ID NO:24) |
| and | |
| TCAAGCACGGCTTGTGTG | (SEQ ID NO:25) |
| Reverse 5′-3′: | |
| GCAGCAATGACGCTCGG; | (SEQ ID NO:3) |
| GGATCAGGTAGGGATACCCGCT; | (SEQ ID NO:11) |
| TTGCAGAATTCAGTGAATCATCG | (SEQ ID NO:12) |
| AGCGGGTATCCCTACCTGAT; | (SEQ ID NO:26) |
| CGATGATTCACTGAATTCTGCA; | (SEQ ID NO:27) |
| AATGACGCTCGGACAGG; | (SEQ ID NO:28) |
| TCAGCGGGTATCCCTACCT | (SEQ ID NO:29) |

In the preceding examples, terms “forward” and “reverse” are use merely to represent directions in reading nucleic acid sequences and are not meant to define nucleic acid fragments of a primer or of an amplification product. Any primer, as contemplated in the present invention, may be translated to be a forward or reverse primer. A PCR™ product is defined by selection of a primer set.

Deoxyribonucleic acid sequences of 5.8S ribosomal RNA are aligned to determine positions or segments of nucleic acid matches and mismatches among species belonging to Aspergillus. Genus-specific oligonucleic acid primers are designed from regions of a DNA segment containing DNA identity among all species compared, even though the regions between the primers may not have a similar nucleic acid composition. The size of the pre-determined fragment is estimated to be from approximately 100 through 600 base pairs, depending on the species. None of the primer combinations set forth herein has DNA identity to yeast, eukaryotic or viral DNA sequences.

In one embodiment of the present invention, primers were created based on complementarity to the 5.8S ribosomal RNA gene of Aspergillus fumigatus, with e.g., A. fumigatus, A. flavus, A. terreus, A. niger and others, as well as in certain other infectious moulds including those belonging to the Fusarium species and Scedosporium species. Of these, primers corresponding to SEQ ID NO:2 (TTGGTTCCGGCATCGA) or SEQ ID NO:3 (GCAGCAATGACGCTCGG) have been used in the methods described herein. These sequences were selected to avoid significant homology to and thus amplification of yeast or human DNA. These specific primers were selected by screening of multiple primers for optimum results. Primers representing shortened or lengthened versions of SEQ ID NO:2 and SEQ ID NO:3 are also contemplated. In some embodiments, primers may be designed to amplify a portion of the nucleic acids that are designated species-specific. Amplification of such nucleic acid fragments may be useful to identify particular species within a genus. For example, primers corresponding to SEQ ID NO:7 (CAGCCGACACCCAACTTTA) or SEQ ID NO:8 (TCCTCCGCTTATTGATATGCTT) were created based on complementarity to the nucleic acid of the 5.8S ribosomal RNA gene of Aspergillus fumigatus.

The present invention may also be performed using a variety of other suitable primers. For example, one can use primers that are complementary to the gene product of a Fusarium species such as that represented by GenBank ID:AF178419 (SEQ ID. NO:5) which corresponds to F. solani 5.8S ribosomal RNA gene, or a fragment thereof, or that of Scedosporium species such as that represented by GenBank ID: AF022485 (SEQ ID. NO:6), as long as these primers are specific for detecting the infectious organism and do not amplify human or other yeast sequences.

Furthermore, one of skill in the art will also recognize that a primer designed to be complementary to other fragments of the gene described in GeneBank ID:AF138288 (SEQ ID NO:1) may be used as a primer for detecting the presence of A. fumigatus in a sample as long as this does not co-amplify human or yeast DNA. Alternatively, one could use primers based on the sequence of any other pathogen, including, A. flavus, A. terreus, A. niger, A. vesicularis, A. nidulans, F. chlamydosporum, F. solani and S. prolificans species.

The primers may be prepared by oligonucleotide synthesis according to standard methods. See, for example, Itakura and Riggs (1980). Additionally, U.S. Pat. Nos. 4,659,774, 4,816,571, 5,141,813, 5,264,566, 4,959,463, 5,428,148, 5,554,744, 5,574,146, 5,602,244, each of which is incorporated herein by reference, describe methods of preparing oligonucleotides. In addition, primers are available commercially at affordable rates. Primers may also be synthesized by recombinant methods using products incorporating viral and bacterial promoters available from Promega (Madison, Wis.).

The use of a primer of between 13 and 100 nucleotides, preferably between 17 and 35 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In accordance with some embodiments, optimal results have been obtained using primers which are identical in length and sequence to the primers described above. However, a person of ordinary skill in the art will recognize that a shortened or lengthened version of a primer may be made to maintain the specificity of the PCR™ amplification and the efficacy of the present inventive method. In some cases, the present invention contemplates that shorter primers containing at least approximately 5 consecutive bases of the nucleic acids sequences of these primers may be suitable. The exact upper or lower limit of the length of the primer is not critical. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 15 to 30 nucleotides, or even longer where desired.

In some embodiments, the primers may be labeled as described below for probes or by other methods known in the art.

In some embodiments, the primers may be used in conjunction with probes described below. However, using primers without probes do not compromise the sensitivity or selectivity of the invention. For example, species-specific probes and primers may be used to identify a particular species, but the use of species-specific primers without the use of species-specific probes may also work as well.

The present invention contemplates minor changes or conservative alterations to a sequence of the primer that do not substantially alter its ability to anneal to its specific target DNA and prime extension during PCR™. For example, any particular nucleic acid, or plurality of nucleic acids, of a primer may be substituted for alternative nucleic acids, which may not allow for Watson-Crick base-pairing at the particular site of alteration on annealing of the primer to the template DNA during PCR™, but nonetheless does not substantially affect the ability of the primer to prime extension during PCR™. Such an alternative primer may be referred to as a variant of the primer(s) described herein. Such a variant primer may be adapted to anneal under appropriate conditions for such variants. For example, the use of a PCR™ reaction buffer having 2-7 mM MgCl₂ or a variation of the annealing temperature between 45° C. and 65° C. may be appropriate for most variants.

In most embodiments, primers may be capable of amplifying a sequence of Aspergillus, Fusarium, or Scedosporium. New species, e.g., new Aspergillus species, are routinely discovered for which primers are designed that are capable of annealing to a new or unknown species having a region of a DNA segment that is complementary to the primer. Therefore, each independently or as a primer set is capable of amplifying any nucleic acids sequence containing a segment of DNA that is complementary to the primer or primers. It is appreciated in the art that variations may be suitable for PCR™, for example, an addition of a nucleic acid at the 5′ or 3′ end of the primer providing additional complementation to the DNA template may be suitable. A substitution of one or more nucleic acid for another within a primer may also be suitable.

The optimum primer concentration, as well as the optimum primer identity, must be determined empirically by testing. Primer concentrations that are too low may result in little or no PCR™ product, while concentrations that are too high may result in amplification of non-target sequences.

Generally, only one PCR™ assay, using a single primer set, will be needed in order to identify the species of Aspergillus, Fusarium, or Scedosporium present within a sample. However it will be appreciated that there may be a time where a parallel PCR™ assay, using a second primer set, may be utilized in order to further clarify the identity of a species present within a sample. Similarly, upon optimization of PCR™ conditions multiple novel primer sets may be used in a single PCR™ assay.

While the novel primers disclosed herein have been designed to enable PCR™ amplification of regions of 5.8S rDNA, it will be appreciated that they may be also applied, individually or in combination, to various other applications. For example, they may be used as molecular probes, or primers for alternative molecular techniques.

2. Probe Design and Synthesis

The invention further utilizes probes for the identification as well as the real-time detection and/or quantitation of a mould DNA such as an invasive mould DNA. As well known in the art, a probe is a nucleic acid molecule that is complementary to a target sequence, which is exemplified in the present invention by a DNA sequence that is specific or unique to an invasive mould. Nucleic acid sequences that are “complementary” are those that are capable of base-pairing according to the standard Watson-Crick complementary rules. As used herein, the term “complementary sequences” means nucleic acid sequences that are “essentially complementary”, as may be assessed by the same nucleotide comparison set forth above, or as defined as being capable of hybridizing to the nucleic acid segment under relatively stringent conditions such as those described herein. Alternatively the probes may be “substantially complementary”, wherein the sequence of the probe is complementary to 80%, 85%, 90%, 95%, or 99% but is still capable of base-pairing to a nucleic acid sequence of an invasive mould DNA sequence.

Probes may be designed to be genus-specific, or species-specific. A genus-specific probe is one which may be complementary to nucleic acids within a particular genus or selected genera. In a non-limiting example, a genus-specific probe may be complementary to nucleic acid fragments of Aspergillus, Fusarium and Scedosporium genera, represented by SEQ ID NO:1, SEQ ID NO:5, SEQ ID NO:6, respectfully, or fragments thereof. In one particular embodiment, a genus-specific probe of the invention has the sequence TGAAGAACGCAGCGAAATGCGATAA (SEQ ID NO:4). A species-specific probe is one which may be complementary to nucleic acids of a particular or selected species. For example, a species-specific probe may be complementary to a nucleic acid fragment encoding Aspergillus fumigatus, as represented by SEQ ID NO:1. Examples of species-specific probes (SEQ ID NO:17, 18, 19, 20 and 21) which may be appropriate in the present invention include species-specific probes such as A. fumigatus: 5′-TTCTAAGGTTGACCTCGGATCAGGTAGG-3′ (SEQ ID NO:18); A. fumigatus: 5′-CAGCCGACACCCAACTTTATTTTTCTAAGGT-3′ (SEQ ID NO:19); A. niger: 5′-CTGCCGACGTTTTCCAACCATTCTT-3′ (SEQ ID NO:20); A. flavus: 5′-TTGCCGAACGCAAATCAATCTTTTTC-3′ (SEQ ID NO:21); A. terreus: 5′-CCGCCGACGCATTTATTTGCA-3′ (SEQ ID NO:17)

As probes are used as hybridizing fragments, they should be of sufficient length to provide specific hybridization to a RNA or DNA. The use of a hybridization probe of between about 10-14, or 15-20, or 10-50, or about 100 nucleotides in length allows the formation of a duplex molecule that is both stable and selective. Molecules having complementary sequences over stretches greater than 20 bases in length are generally preferred, in order to increase stability and selectivity of the hybrid, and thereby improve the quality and degree of particular hybrid molecules obtained.

In accordance with some embodiments of the present invention, optimal results have been obtained using probes which are identical in length and sequence to the probes described herein. However, a person of ordinary skill in the art will recognize that a shortened or lengthened version of a probe may be made to maintain the specificity of the PCR™ amplification and the efficacy of the present inventive method. In some cases, the present invention contemplates that shorter probes containing at least approximately 10 consecutive bases of the nucleic acid sequences may be suitable. The exact upper or lower limit of the length of the probe is not critical. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 15 to 30 nucleotides, or even longer where desired.

Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 300, 500, 600, 700, 800, and longer are contemplated as well.

The present invention contemplates minor changes or conservative alterations to a sequence of the probe that do not substantially alter its ability to hybridize to its specific target DNA. For example, any particular nucleic acid, or plurality of nucleic acids, of a probe may be substituted for alternative nucleic acids, which may not allow for Watson-Crick base-pairing at the particular site of alteration on hybridizing of the probe to the nucleic acid, but nonetheless does not substantially affect the ability of the probe to hybridize. Such an alternative probe may be referred to as a “variant” of the probe described herein.

In most embodiments, genus-specific probes may be capable of hybridizing a sequence of Aspergillus, Fusarium, or Scedosporium. New species, e.g., belonging to Aspergillus, are routinely discovered for which probes capable of annealing to a new or unknown species having a region of a DNA segment that is complementary to the probe are designed. Therefore, each probe, independently or as a set, is capable of hybridizing. to any nucleic acid sequence containing a segment of DNA that is complementary to the probe or probes. For example, an addition of a nucleic acid at the 5′ or 3′ end of the probe providing additional complementation to the DNA template may be suitable. In other instances, a substitution of one or more nucleic acids for another within a probe may also be suitable.

The probes of the present invention may be made by methods well known in the art, such as chemical synthesis or by recombinant methods as set forth above for primers.

In some embodiments, probes of the present invention may be labeled, such as with fluorescent compounds, radioactive isotopes, antigens, ligands such as biotin-avidin, colorimetric compounds, or other labeling agents known to those of ordinary skill in the art, to allow detection and quantification of amplified DNA. The use of fluorescent labels is especially contemplated in the real-time PCR™ methods of the present invention.

In some embodiments, the probe may contain an oligonucleotide with a 5′ reporter dye and a 3′ quencher dye. A fluorescent reporter dye, such as 6-carboxyfluorescein (FAM™) is covalently linked to the 5′ end of an oligonucleotide. TET™ (tetrachloro-6-carboxyfluorescein), JOE™ (2,7, -dimethoxy-4,5-dichloro-6-carboxyfluorescein, VIC™ and HEX (hexachloro-6-carboxyfluorescein) have also been used as reporter dyes. Each of the reporter is quenched by a fluorescent quencher, such as TAMRA™ (6-carboxy-N,N,N′,N′-tetramethylrhodamine) or a non-fluorescent quencher, such as Black Hole Quencherm™ attached via a linker that is usually at the 3′ end. A passive reference, such as ROX™, is used as an internal reference to which the reporter-dye signal can be normalized during data analysis. In some embodiments, the use of SYBR® Green, which does not require a fluorescent probe, can be a desirable option, especially for assays involving a large number of primers and/or probes.

Other examples of fluorescent labels include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein, Oregon Green 488, Oregon Green 500, Oregon Green 514, Pacific Blue, REG, Rhodamine Green, Rhodamine Red, ROX, and Texas Red. Alternatively, enzyme tags such as urease, alkaline phosphatase or peroxidase may be used. In the case of enzyme tags, calorimetric indicator substrates are known that can be employed to provide a detection means that is visibly or spectrophotometrically detectable, to identify specific hybridization with complementary nucleic acid containing samples.

The use of highly specific primers and fluorescent probe sequences are designed to yield target amplicons to unique regions of the invasive pathogenic mould genome. Thus, the inventors contemplate that rapid real-time PCR™-based diagnostic methods as described herein will allow for appropriate aggressive treatment regimens to be designed to treat the invasive mould.

3. Hybridization

Accordingly, the primer and probe nucleotide sequences of the invention may be used for their ability to selectively form duplex molecules with complementary stretches of DNAs and/or RNAs or to provide primers for amplification of DNA or RNA from samples or as probes to detect specific sequences in the amplified DNA. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

A medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results. In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In general, it is envisioned that the probes or primers described herein will be useful as reagents in solution hybridization, as in PCR™ or other amplification methods, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the specification are incorporated herein by reference.

4. Amplification by PCR™

A number of template dependent processes are available to amplify the pathogenic mould DNA in a given biological sample as described herein. One of the best known amplification methods in the polymerase chain reaction (referred to as PCR™) which is described in detail in U.S. Pat. Nos. 4,683,202 and 4,800,159, and in Innis et al., 1990. Briefly, in PCR™, two primer sequences are prepared that are complementary to regions on opposite complementary strands of the marker sequence. An excess of deoxynucleoside triphosphates are added to a reaction mixture along with a heat-stable DNA polymerase, e.g., Taq polymerase or Vent polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

The reverse transcriptase (RT) PCR™ amplification procedure is a variant of PCR™ that permits amplification of mRNA templates. Thus, one may amplify the invasive mould DNA from a biological sample, by first isolating the mRNA and reverse transcribing this utilizing for example a RT-PCR kit (Invitrogen), according to the manufacturer's instructions.

It is understood that a complementary sequence is one which is capable of forming complement, in which an adenine pairs with a thymine or a guanine pairs with a cytosine. The conditions of PCR™ is carefully controlled so that the primers will hybridize preferentially to the desired target sequence throughout each amplification cycle, thereby limiting the synthesis of undesirable DNA segment located beyond any PCR™ primer or outside any primer pair. In an ideal case, each primer would be able to bind only to a desired target in the initial template. Each primer is typically used as a member of a primer pair, including a 5′ upstream primer that hybridizes with the complementary 3′ end of the nucleic acid template to be amplified and a 3′ downstream primer that hybridizes with the complementary 5′ end of the nucleic acids template to be amplified.

One of ordinary skill in the art will understand the term “complementary” as used herein, to mean “wholly complementary” or “substantially complementary,” for example, a primer may be less than 100% complementary to its target template sequence but is still capable of annealing thereto in a specific manner under appropriate conditions.

Generally, “DNA identity” is used to refer to a region of a DNA of a species or organism that is similar or substantially similar to a region of a DNA of another species or organism in the arrangement of nucleic acids. In some embodiments, a region that contains a DNA identity among species within a particular genus is referred to as a conserved region at the genus level and is referred to as “genus-specific.” Additionally, in some embodiments, a region that contains a DNA identity among species and among genera within a particular family is referred to as a conserved region at the family level. In some cases, a region of a DNA may be conserved within a class, order, phylum or kingdom classification. “Primer set,” as used in some instances, is referred to as a pair or multiple primers designed for amplifying a particular region.

Each PCR™ is performed with at least one monospecific control sample or standard of known species identity (e.g., Aspergillus funigatus). It will be appreciated that controls containing more than one known species may be entertained. For example, positive controls may include nucleic acid templates of mould, or negative controls may exclude such templates. It will be appreciated that other controls routinely used in the art may also be employed. The nucleic acid templates of the controls or standards of known species may be obtained from a source such as the American Tissue Culture Collection or grown in laboratory and purified according to techniques well known in the art.
a. Theoretical Considerations

PCR™ is characterized by three phrases. In the exponential phase, the number of molecules of the amplified target DNA increase by a factor approaching two with every cycle of the reaction until some reagent becomes limiting. If a graph is plotted in which the cycle number is on the X axis and the log of the concentration of the amplified target DNA is on the Y axis, a curved line of characteristic shape is formed by connecting the plotted points. The exponential phase is represented by a positive slope of the line and the first phase of PCR™. The reaction in the exponential phase is very specific and precise, with product accumulating at every cycle. In the linear phase, the rate of amplification becomes increasingly diminished. The reaction components are consumed, reagents become limiting and products begin to degrade. In the plateau phase, the slope of the line begins to decrease and eventually becomes zero. At this point the concentration of the amplified target DNA becomes asymptotic to some fixed value.

With traditional methods of PCR™, products are detected from the plateau phase. In some embodiments, the present invention relies on quantitative PCR™ to detect the presence of an infectious mould in a sample. Quantification of PCR™ products from the plateau phase is difficult because some reactions with different amount of starting materials may have the same plateau phase. Thus, the data may not represent the initial amount of starting materials. The linear phase is too highly variable for any meaningful measurements of PCR™ products. The depletion will occur at different rates for each replicate. Real-time PCR™ detects product accumulation during the exponential phase of the reaction. The exponential phase is ideal for quantifying PCR™ products because amplification is directly proportional to the starting concentration of the target before the reaction began. By determining the concentration of the amplified products of the target DNA in PCR™ reactions that have completed the same number of cycles and are in their exponential ranges, it is possible to determine the relative concentrations of the specific target sequence in the original DNA mixture.

Real-time PCR™ quantitation eliminates post-PCR processing of PCR™ products. This helps to increase throughput, reduce the chances of carryover contamination and remove post-PCR processing as a potential source of error. In comparison to conventional PCR™, real-time PCR™ also offers a much wider dynamic range of approximately up to 10⁷-fold. Conventional PCR™ offers dynamic range of approximately 1000-fold. This means that a wide range of ratios of target and normalizer can be assayed with equal sensitivity and specificity. The broader the dynamic range, the more accurate the quantitation. Generally, the sensitivity of real-time PCR™ allows detection of the target in the range of 2 pg of DNA or total RNA.
b. Real Time PCR™

In some embodiments, the present invention utilizes quantitative real-time PCR-based methods to detect the presence of infective mould DNA in a biological sample. RNA or DNA, isolated from biological samples suspected of containing mould, may be amplified and simultaneously quantitated using the real-time PCR™ technique of the present invention.

Real-time PCR™ refers to an ability to monitor progress of PCR™ by a point in time during cycling whereby PCR™R products can be detected as they accumulate, rather than after a fixed number of cycles. Higuchi et al. (1992) pioneered real-time PCR™ by introducing an intercalator ethidium bromide dye in each amplification reaction, irradiating the sample with ultraviolet light and detecting the resulting fluorescence with a CCD camera. The principal drawback to an intercalator-based detection is that both specific and non-specific PCR™ products generate signals. Holland et al. (1991) removed this drawback by demonstrating that cleavage of a target probe during an amplification reaction by the 5′ nuclease activity of Taq DNA polymerase could be used to detect amplification of the target-specific product. The 5′ exo-nuclease activity of the enzyme acts upon the surface of the template to remove obstacles downstream of the growing amplicon that may interfere with its' generation. The 5′ nuclease activity makes it possible to detect PCR™ amplification in real-time. Lee et al., (1993) eliminated the need for post-PCR processing by coupling nuclease activity to a quenching activity between a reporter dye and a quencher dye attached to the probe. The improvements of Higuchi et al. (1992), Holland et al. (1991) and Lee et al. (1993) have resulted in the real-time PCR™ technique used in the present invention. The advantage of the fluorogenic probe over the DNA binding dye method is that specific hybridization between probe and target is required to generate a detection signal. However, the art lacks confirmation that real-time methods may be useful in the clinical setting for the diagnosis of mould infection.

Recent advances in fluorescence measurements using the light-emitting diode (LED) technology has led to the development of instrumentation coupled with PCR™ technology for real-time detection of specific PCR™ products. This real-time PCR™ technology offers several advantages over conventional PCR™ methods, such as a lower risk of PCR™ contamination, shorter turnaround time, and quantitative PCR™ analysis. Some of the most popular commercially available systems coupling PCR™ technology with real-time detection of PCR products include the ABI PRISM 7700 Sequence Detection System (PE Applied Biosystems, Foster City, Calif., USA), the LightCycler™ (Idaho Technologies, Idaho Falls, Ind., USA; and Roche Diagnostics, Indianapolis, Ind., USA), and the Smart Cycler® (Cepheid, Sunnyvale, Calif., USA).

TaqMan™ PCR (Perkin-Elmer Corp., Applied Biosystems, Foster City, Calif.) is an approach that combines PCR, probe hybridization and signal generation in one step. The TaqMan™ probe consists of a reporter dye with a fluorescein derivative at the 5′ end and a 3′ quencher dye. The fluorescence emission of the reporter dye is suppressed in the intact probe by Forster-type energy transfer. During amplification, the probe is cleaved by the 5′ nuclease activity of the DNA polymerase only when it is hybridized to a complementary target. When probe-specific PCR™ probe has been generated, an increase in reporter dye fluorescence, resulting from the cleavage between the reporter and quencher, occurs. The amount of reported dye released is proportional to the amount of DNA amplified by PCR™. TaqMan™ fluorescence assay enables samples to be analyzed as soon as PCR™ is completed. No post-amplification manipulation is required.

A quantitative PCR™ assay with the LightCycler (Roche Diagnistics, Mannheim, Germany) combines rapid thermocycling with glass capillaries with online fluorescence detection of the PCR amplicon. Similar to the TaqMan method, this detection system is based on fluorescence resonance energy transfer with two different specific oligonucleic acids. The first hybridization probe is labeled with fluorescein, while the second hybridization probe is labeled with the fluorophore LightCycler Red 640. Both probes can hybridize in a head-to-tail arrangement, bringing the two dyes into close proximity. A transfer of energy between the two probes results in emission of red fluorescence light, which is measured by photohybrids. The level of fluorescence is proportional to the amount of DNA generated during the PCR™ process.

The real-time PCR™ system is based on the detection and quantitation of a fluorescent reporter. This signal increases in direct proportion to the amount of PCR™ product in a reaction. By recording the amount of fluorescence emission at each cycle, it is possible to monitor the PCR™ reaction during exponential phase where the first significant increase in the amount of PCR™ product correlates to the initial amount of target template. The chemistry of real-time PCR™ occurs primarily during the exponential phase and is further described below.
c. Chemistry in Real-time PCR

The chemistry of real-time PCR™ occurs primarily during extension steps of the exponential phase. An intact probe has a specific distance between the reporter dye to the quencher dye which results in suppression of the reporter fluorescence, primarily by Forster-type energy transfer (Lakowicz, 1983; Foster, 1948). Hence, there is no fluorescence signal if the probe does not bind to its target. When a high energy dye is in close proximity to a low-energy dye, there is a transfer of energy from high to low, suppressing fluorescence signals. During PCR™, if the target template is present the probe specifically anneals to the template, usually between the forward and reverse primer sites, to form a probe-template duplex. When the probe forms and maintains a duplex formation with the template, the proximity of the reporter dye to the quencher dye also results in suppression of the reporter fluorescence. In the process of polymerization of the strand, primer extension will eventually encounter the probe-template duplex. DNA polymerase containing 5′-3′ nucleolytic activity, such as AmpliTaq Gold™ cleaves the probe, displacing the probe from the target. The enzyme will only cleave the probe between the reporter and the quencher only if the probe is in a probe-template duplex. The polymerase does not digest free probe. Once the probe is displaced, the distant between the reporter and quencher dye changes, eliminating the quenching ability of the quencher. The reporter then emits a fluorescence signal. At the same time, the cleavage removes the probe from the target strand and allows for primer extension to continue to the end of the template strand.

Additional reporter dye molecules are cleaved from their respective probes with each cycle, effecting an increase in fluorescence intensity proportional to the amount of amplicon produced. This process does not interfere with the exponential accumulation of product. The separation of the reporter dye from the quencher dye results in an increase of fluorescence of the reporter. The increase in fluorescence is measured, and is a direct consequence of target amplification during PCR™. Accumulation of PCR™ products is detected by monitoring the increase in fluorescence of the reporter dye.

The specificity of the method results from the requirement of the enzyme for primer and probe complementary to target in order for amplification and cleavage to occur. The fluorescence signal is generated only if the target sequence for the probe is amplified during PCR™. Because of these requirements, any non-specific amplification is not detected.

Reporter dyes commonly used in real-time PCR™ are SYBR™ green, TaqMan™ and Molecular Beacons. SYBR green binds to double stranded DNA, which may lead to overestimation of the target concentration by binding to primer-dimer and non-specific reactions. TaqMan™ probes are oligonucleotides that contain a fluorescent dye, typically on the 5′ base, and a quenching dye, typically located at the 3′ base. In some embodiment, TaqMan™ probes are used as reporter and quencher dyes. TaqMan dyes commonly used in the analysis are FAM, TET, JOE, HEX, TAMRA, and ROX. Molecular Beacons also contain fluorescent and quenching dyes, but fluorescence resonance energy transfer (FRET) only occurs when the quenching dye is directly adjacent to the fluorescent dye. Molecular beacons are designed to adopt a hairpin structure while free in solution, bringing the fluorescent dye and quencher in close proximity. When a molecular beacon hybridizes to a target, the fluorescent dye and quencher are separated, FRET does not occur, and the fluorescent dye emits light upon irradiation. Unlike TaqMan™ probes, molecular beacons are designed to remain intact during the amplification reaction, and must rebind to the target in every cycle for signal measurement. Some embodiments of the present invention may be adapted to use molecular beacons as reporter probes.

In some embodiments, the ABI Prism TM Sequence Detection System (PE Applied Biosystem) is used to monitor the increase of the reporter fluorescence following PCR. Reporter signal is normalized to the emission of a passive reference. The results are defined as ΔRn, the difference between the normalized reporter fluorescence in the sample and the No Template Control tubes.

Multiplex PCR refers to the use of more than one primer pair in the same tube. In an embodiment, relative quantitation may be performed in a single tube by having one primer pair amplifying the target and another primer pair amplifying the reference. Competition may be avoided by designing primers to amplify two different segments in the same reaction tube and by limiting the concentrations of primers. In some embodiments, species determination may be performed in a single reaction tube by having multiple primer pairs amplifying different segments in the same reaction tube. Multiple reporter dyes having non-overlapping wavelength maxima may be used to distinguish one target from another.

Multicomponenting refers to the use of multiple reporter dyes in a single reaction tube to monitor more than one amplification. Each individual dye makes up a distinct fluorescence spectrum. This spectrum represents one-fluorescent reading from one well. A combination of dyes with a large difference in emission maxima, such as FAM (target) and VIC (endogenous control), is suitable for discrimination of contribution from each dye. Reporter dyes that are commonly used for multicomponent analysis are FAM, TET, JOE, HEX. TAMPA is reserved as the quencher on the probe and ROX as the passive reference. In some embodiments, multiple probes with a combination of optimal dyes may be used to determine the presence of particular mould species.

5. Quantification of a PCR™ Product

In some embodiments, the present invention utilizes quantitative real-time PCR-based methods to detect the presence of infective mould DNA in a biological sample. RNA or DNA, isolated from biological samples suspected of containing mould, may be amplified and simultaneously quantitated using the real-time PCR™ technique. This technique entails the use of a fluorogenic (or otherwise labeled) hybridization probes or dsDNA-specific fluorescent dyes to detect PCR™ product during amplification (real-time detection) without purification or separation by gel electrophoresis. The sensitivity of the probe(s) obtained by this method, allows measurement of the PCR™ product during the exponential phase of amplification before the critical reactants become limiting. This method does not require the separation of the PCR™ products.

Quantification refers to an ability to measure the amount of target in unknown samples. A common quantification method used in conventional end-point PCR™ is estimating band sizes from gel electrophoresis. Another method is quantitative competitive PCR™ whereby a competitor amplicon is constructed that contains the same primer binding sites and has the same amplification efficiency as the target, but is somehow distinguishable from the target. Gel electrophoresis is used to discriminate between the two products. With real-time PCR™, the need to have a competitor amplicon with the target is eliminated. The dynamic range of quantitation is greatly expanded because data is collected for every cycle of PCR™, rather than at the endpoint.

Accordingly, the threshold cycle (C_{T}) is defined as the fractional cycle number at which the reporter fluorescence generated by cleavage of the probe passes a fixed threshold above baseline. The threshold is the level of detection or point at which a reaction reaches a fluorescent intensity of above background. The threshold line is set in the exponential phase of the amplification. The C_{T} value is the cycle at which a significant increase in Delta Rn is first detected. Rn⁺ is the Rn value of a reaction containing all components, Rn⁻ is the Rn value of an unreacted sample (baseline value or the value detected in NTC). Delta Rn is the difference between Rn⁺ and Rn⁻. It is an indicator of the magnitude of the signal generated by the PCR™. The threshold cycle is when the system begins to detect the increase in the signal associated with an exponential growth of PCR™ product during the log-linear phase. This phase provides the most useful information about the reaction (certainly more important than the end point). The slope of the log-linear phase is a reflection of the amplification efficiency. For the slope to be an indicator of real amplification (rather than signal drift), there has to be an inflection point. This is the point on the growth curve when the log-linear phase begins. It also represents the greatest rate of change along the growth curve. (Signal drift is characterized by gradual increase or decrease in fluorescence without amplification of the product.) The important parameter for quantitation is the C_{T}. The higher the initial amount of genomic DNA, the sooner accumulated product is detected in the PCR™ process, and the lower the C_{T} value. The choice of threshold, which will determine the C_{T} value is up to the user and one of the subjective elements in real-time PCR™. It should be placed above any baseline activity and within the exponential increase phase (which looks linear in the log transformation). Usually, system-integrated software allows determination of the cycle threshold by a mathematical analysis of the growth curve. This provides better run-to-run reproducibility. A C_{T} value of 40 may mean no amplification. Besides being used for quantitation, the C_{T} value can be used for qualitative analysis as a pass/fail measure.

Absolute quantification refers to an expression of quantity in terms of an absolute value. The method requires the standards to be known by some independent means. For example, concentration of nucleic acids may be determined by A₂₆₀ and converted into the number of copies by using molecular weight. Plasmid DNA or in vitro transcribed RNA are commonly used to prepare absolute standards. Unknown samples are quantitated by interpolating their quantity from a standard curve. In some embodiments, absolute quantitation may be performed.

Relative quantitation refers to an expression of quantity relative to some basis sample, such as the calibrator. The calculation methods used for relative quantitation are the standard curve method and the comparative C_{T} method. In the standard curve method, the target quantity is determined from the standard curve and expressed as an n-fold difference relative to the calibrator quantity. In some embodiments, relative quantitation using either the standard curve or the comparative method may be performed.

Conditions for specific hybridization of primers and probes to particular template targets is determined empirically, by varying the annealing temperature in several degree increments and comparing the specificity and efficiency of the amplification process.

Amplification is conducted according to procedures in the art to which this invention relates and such as described in U.S. Pat. No. 4,683,202. According to some embodiments of the present invention, the amplification reaction mixture may include 50 nM-200 μM of each primer, 100-200 μM each dNTP, 2-7 mM MgCl₂, or 1-2 U GoldTaq DNA polymerase (ABI). In some embodiments, PCR™ cycling may be performed under the following conditions: denaturation at a temperature of 92° C.-96° C. for 20-60 seconds, annealing at a temperature from 55° C. to 65° C. for 20-120 seconds and extension at a temperature of 63° C.-75° C. for 20-120 seconds. In some embodiments, an initial incubation period of 10-20 minutes at a temperature of 92° C.-96° C. may be used to activate the enzyme. Also in some embodiments, a final incubation period of approximately 1-5 minutes may be used to facilitate synthesis of any frayed end-fragment. In most cases, between 20 and 45 cycles may be performed. For example, the following PCR™ conditions (Table 1) may be pairs of primers of SEQ ID NO:1 and SEQ ID NO:2 and a probe of the invention:

TABLE 1

| | | | | | Additional |
|---|---|---|---|---|---|
| Initial | Denaturation | Annealing | Extension | Cycles | Activation |
| 92° C., 15 min | 96° C., 30 sec | 60° C., 60 sec | 72° C., 60 sec | 40 | 72° C., 300 sec |
| 95° C., 15 min | 96° C., 40 sec | 63° C., 45 sec | 70° C., 45 sec | 45 | 72° C., 300 sec |
| 95° C., 15 min | 95° C., 20 sec | 60° C., 20 sec | 72° C., 20 sec | 40 | 72° C., 300 sec |

It will be appreciated by those of ordinary skill in the art that the PCR™ conditions provided herein are merely examples and may be varied so as to optimize conditions where, for example, alternative PCR™ cycler or DNA polymerase are used, where the quality of the template DNA differs, or where variations of the primers specifically exemplified herein are used, without departing from the scope of the present invention. The PCR™ conditions may be altered or optimized by changing the concentration of the various constituents within the reaction, altering the number of amplification cycles, the denaturation, annealing or extension times or temperatures, or the quantity of template DNA, for example. It will be realized that the specificity of the annealing is most important in the first several amplification cycles. Those skill in the art will appreciate there are a number of other ways in which PCR™ conditions may be optimized to overcome variability between reactions.

It will be understood that where no specificity is exemplified herein appropriate PCR™ annealing temperatures for any primers within the scope of the present invention may be derived from the calculated melting temperature of that primer. Such melting temperature may be calculated using standard formulas, such as that described in Sambrook, 2001. As will be understood by those of ordinary skill in the art to which this invention relates annealing temperature may be above or below the melting temperature but generally an annealing temperature of approximately 0° C. to 10° C. below the calculated melting temperature of the primer may be suitable.

The reverse transcriptase (RT) PCR™ amplification procedure is a variant of PCR™ that permits amplification of mRNA templates. Thus, one may amplify the invasive mould DNA from a biological sample, by first isolating the mRNA and reverse transcribing this utilizing for example a RT-PCR kit (Invitrogen), according to the manufacturer's instructions. In some embodiment, real-time RT-PCR performs reverse transcription and PCR™ in a single buffer system or in two steps, which are performed separately in different tubes.

6. Other Amplification Procedures

A number of other template dependent processes are available to amplify the oligonucleotide sequences. For example, the ligase chain reaction (“LCR”), disclosed in European Application No. 320308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2,202,328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et aL., 1989; Gingeras et al., PCT Application WO 88/10315, incorporated herein by reference in their entirety). European Application No. 329822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR™” (Frohman, 1994; Ohara et al., 1989).

K. Methods for Detection of Nucleic Acids

1. Energy Transfer

Another emerging method for detecting nucleic acids involves energy transfer. Labeling hybridization oligonucleotide probes with fluorescent labels is a well known technique in the art and is a sensitive, non-radioactive method for facilitating detection of probe hybridization. More recently developed detection methods employ the process of fluorescence energy transfer (FET) rather than direct detection of fluorescence intensity for detection of probe hybridization. FET occurs between a donor fluorophore and an acceptor dye (which may or may not be a fluorophore) when the absorption spectrum of one (the acceptor) overlaps the emission spectrum of the other (the donor) and the two dyes are in close proximity. Dyes with these properties are referred to as donor/acceptor dye pairs or energy transfer dye pairs. The excited-state energy of the donor fluorophore is transferred by a resonance dipole-induced dipole interaction to the neighboring acceptor. This results in quenching of donor fluorescence. In some cases, if the acceptor is also a fluorophore, the intensity of its fluorescence may be enhanced. The efficiency of energy transfer is highly dependent on the distance between the donor and acceptor, and equations predicting these relationships have been developed (Forster, 1948). The distance between donor and acceptor dyes at which energy transfer efficiency is 50% is referred to as the Forster distance (R_{O}). Other mechanisms of fluorescence quenching are also known including, for example, charge transfer and collisional quenching.

Energy transfer and other mechanisms which rely on the interaction of two dyes in close proximity to produce quenching are an attractive means for detecting or identifying nucleotide sequences, as such assays may be conducted in homogeneous formats. Homogeneous assay formats are simpler than conventional probe hybridization assays which rely on detection of the fluorescence of a single fluorophore label, as heterogeneous assays generally require additional steps to separate hybridized label from free label. Several formats for FET hybridization assays are reviewed in Binninger, et al., (1992).

Homogeneous methods employing energy transfer or other mechanisms of fluorescence quenching for detection of nucleic acid amplification have also been described. Higuchi et al. (1992 and 1993) disclosed methods for detecting DNA amplification in real-time by monitoring increased fluorescence of ethidium bromide as it binds to double-stranded DNA. The sensitivity of this method is limited because binding of the ethidium bromide is not target specific and background amplification products are also detected. Lee et al. (1993) disclose a real-time detection method in which a doubly-labeled detector probe is cleaved in a target amplification-specific manner during PCR™. The detector probe is hybridized downstream of the amplification primer so that the 5-3 exonuclease activity of Taq polymerase digests the detector probe, separating two fluorescent dyes which form an energy transfer pair. Fluorescence intensity increases as the probe is cleaved. WO 96/21144 discloses continuous fluorometric assays in which enzyme-mediated cleavage of nucleic acids results in increased fluorescence. Fluorescence energy transfer is suggested for use in the methods, but only in the context of a method employing a single fluorescent label which is quenched by hybridization to the target.

Signal primers or detector probes which hybridize to the target sequence downstream of the hybridization site of the amplification primers have been described for use in detection of nucleic acid amplification (U.S. Pat. No. 5,547,861). The signal primer is extended by the polymerase in a manner similar to extension of the amplification primers. Extension of the amplification primer displaces the extension product of the signal primer in a target amplification-dependent manner, producing a double-stranded secondary amplification product which may be detected as an indication of target amplification. The secondary amplification products generated from signal primers may be detected by means of a variety of labels and reporter groups, restriction sites in the signal primer which are cleaved to produce fragments of a characteristic size, capture groups, and structural features such as triple helices and recognition sites for double-stranded DNA binding proteins.

Many donor/acceptor dye pairs known in the art and may be used in the present invention. These include, for example, fluorescein isothiocyanate (FITC)/tetramethylrhodamine isothiocyanate (TRITC), FITC/Texas Red™ (Molecular Probes), FITC/N-hydroxysuccinimidyl 1-pyrenebutyrate (PYB), FITC/eosin isothiocyanate (EITC), N-hydroxysuccinimidyl 1-pyrenesulfonate (PYS)/FITC, FITC/Rhodamine X, FITC/tetramethylrhodamine (TAMRA), and others. The selection of a particular donor/acceptor fluorophore pair is not critical. For energy transfer quenching mechanisms it is only necessary that the emission wavelengths of the donor fluorophore overlap the excitation wavelengths of the acceptor, i.e., there must be sufficient spectral overlap between the two dyes to allow efficient energy transfer, charge transfer or fluorescence quenching. P-(dimethyl aminophenylazo) benzoic acid (DABCYL) is a non-fluorescent acceptor dye which effectively quenches fluorescence from an adjacent fluorophore, e.g., fluorescein or 5-(2-aminoethyl) aminonaphthalene (EDANS). Any dye pair which produces fluorescence quenching in the detector nucleic acids of the invention are suitable for use in the methods of the invention, regardless of the mechanism by which quenching occurs. Terminal and internal labeling methods are both known in the art and maybe routinely used to link the donor and acceptor dyes at their respective sites in the detector nucleic acid.

2. Luminex

The Luminex technology allows the quantitation of nucleic acid products immobilized on color coded microspheres. The magnitude of the biomolecular reaction is measured using a second molecule called a reporter, and is done on individual microspheres as they flow through a detection chamber. The reporter molecule signals the extent of the reaction by attaching to the molecules on the microspheres. As both the microspheres and the reporter molecules are color coded, digital signal processing allows the translation of signals into real-time, quantitative data for each reaction.

L. Kits

The invention also contemplates kits designed to detect the presence of invasive pathogenic moulds that can cause IMI by the methods described herein. Thus, in some embodiments, the kits of the invention will comprise one or more components designed to be used in conducting real-time PCR™.

In a non-limiting example, the kits will comprise primers, probes, enzymes for reverse transcription, enzymes for amplification and additional agents such as buffers, nucleotides, water, and suitable positive and negative standards. The primers and probes may be lyophilized or may optionally be provided dissolved in a solvent containing such components as water, Tris, or other components known to those of ordinary skill in the art. The primers and probes may be provided labeled or with reagents that could be used to create labeled probe or primer. The buffers may include full strength or concentrated buffers, which may contain Tris, DMSO and/or other additives. The enzymes include Taq-polymerase or similar thermostable DNA polymerase used by those of skill in the art in conducting PCR, and reverse transcriptase, preferably in solution, such solution containing glycerol and/or water. Other reagents contemplated as useful include magnesium chloride and deoxynucleotides such as dATP, dCTP, dGTP and dTTP.

The kits may also comprise agents for DNA and/or RNA isolation and purification. The components of such kits will thus comprise one or more of these reagents in suitable container means.

The contents of such PCR™ kits and the foregoing compositions may be modified by those of ordinary skill in the art to achieve specific results and such kits and compositions are intended to be part of the present invention.

The components of the kits may be packaged either in aqueous media or in lyophilized form. The suitable container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the reagent containers in close confinement for commercial sale. Such containers may include injection or blow-moulded plastic containers into which the desired vials are retained. Kits of the invention will also typically contain written instructions on the methods of using the kit.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Case Definition and Serum Samples.

As described earlier, cases of IMI were defined according to the criteria established by EORTC and Mycoses Study Group (Ascioglu et al., 2002). Depending on the degree of diagnostic certainty, the cases are defined as “definitive”, “probable”, “possible”, and “unlikely” IMI. “Proven” IMI represented a tissue diagnosis where branched septate hyphae, inflammation, and necrosis were seen microscopically and/or the fungus was successfully cultured from the tissue. Most of the patients in this group had pulmonary IMI and were typically neutropenic and/or immunosuppressed for an extended period of time. They exhibited prolonged pneumonia unresponsive to anti-bacterial therapy with nodular and/or cavitory lesions in the lung radiologically. Of the 13 patients with documented IMI in this study, tissue diagnoses were rendered in 12 patients by surgery or biopsy and one by autopsy. Patients with “probable IMI” typically were immunocompromised, had clinical and radiological features of IMI, and isolation of Aspergillus or other septate moulds twice or more from respiratory specimens (such as sputa, bronchoalveolar lavages, and bronchial washings). Of the 28 patients in this category, 24 lacked tissue confirmation and 4 were autopsied after death later. Though pneumonia was seen in 3 of the autopsy cases, moulds were not observed histologically or cultured microbiologically from the autopsy lung tissue. Thus, the IMI status of the four autopsy cases remained probable. Patients with “possible IMI” were those with atypical pulmonary radiology, pneumonia unresponsive to anti-bacterials, and isolation of mould once (rarely without) from the airway. Patients who did not meet the criteria for possible IMI represented “unlikely IMI.” Most patients with known or suspected (probable and possible) IMI had hematologic malignancies, in contrast to solid tumors in the majority of unlikely IMI patients.

Sera from these patients were collected retrospectively from clinical laboratories from October 1999 to Sep. 2001 and stored at −20° C. until assay. A total of 559 serum samples from 106 patients were collected with single serum in 32 patients, 2 to 29 sera (mean 7) in 73 patients over a span of 1-267 days (median 22), and 7 sera in a single patient over 578 days. Most sera were drawn due to suspected episodes of infection. Thus, for patients with unlikely IMI, being low risk for IMI and other infections, fewer serial sera per patient were drawn (Table 2).

Fungal Strains and DNA.

The mould DNA used as a quantitation standard was purified from A. fumigatus strain AF293 using standard cesium chloride density gradient. Candidal DNA was purified from clinical isolates using standard phenol-chloroform methods.

Serum DNA Extraction.

Serum DNA was extracted using a silica-binding method (Boom et al., 1990), with modifications. Briefly, silica beads (Sigma-Aldrich, St. Louis, Mo.) were pretreated with diluted HCl, selected for particle size (1-10 μm) by serial settling in water, and stored at room temperature after autoclave. After serum (2 ml) was mixed with a lysis buffer (9 ml of 6 M guanidinium isothiocynate-Tris/HCl, pH 6) and incubated (37° C.×30 min), silica sludge (50 μl) was added and mixed thoroughly by vortex. The DNA-bound silica beads were pelleted through centriftigation (6000 rpm×10 min), washed once more with the lysis buffer, twice with ethanol, and finally with acetone, then dried. The beads were resuspended in 50 μL of Tris-EDTA buffer (10 mM, pH 7) and the bound DNA was eluted off the beads through filtration (0.45 μm filter, Alltech, Deerfield, Ill.) and centrifugation (12,000 rpm×1 min). Purified DNA was measured at 260 nm and stored at −20° C.

Design of Primers and Probe.

The sequences of primers and probe were designed based on those of A. fumigatus. After evaluation of several candidate genes, the 5.8S ribosomal RNA gene was chosen for its conservation among various Aspergillus species (A. fumigatus, A. flavus, A. terreus, A. niger and others). The, sequences were selected to avoid significant homology to and thus amplification of yeast and human sequences. Additional guidelines for real-time PCR™ design of primers and probe were also followed (Applied Biosystems, Foster City, Calif.). PCR™ primers for Aspergillus DNA were designed to be in the region of 5.6s RNA gene with common sequence identity between the Aspergillus species fumigatus, terreus, flavus, and niger but differ from Candidaby several nucleotides. The forward primer differs from Candidaby the end nucleotide at the 3′ end and the reverse primer differs by three nucleotides in the middle.

TABLE 2

| | | | |
|---|---|---|---|
| Primer | | | |
| Name | Primer Identity | Position | SEQ ID NO: |

| | | | |
|---|---|---|---|
| F3 | TCAACAACGGATCTCTTGGTT | 220 | 13 |
| F4 | TTGGTTCCGGCATCGA | 235 | 2 |
| F5 | TCAAGCACGGCTTGTGTGT | 378 | 9 |
| F6 | CAACGGATCTCTTGGTTCCG | 224 | 10 |
| R2 | GGATCAGGTAGGGATACCCGCT | 548 | 11 |
| R3 | TTGCAGAATTCAGTGAATCATCG | 286 | 12 |
| R4 | GCAGCAATGACGCTCGG | 359 | 3 |

In pilot studies, a number of primers in various combinations were tested for sensitivity and specificity. The optimal primers were found to be 5′-TTGGTTCCGGCATCGA (SEQ ID. NO:2), and 5′-GCAGCAATGACGCTCGG (SEQ ID. NO:3), corresponding to positions 235 to 376 (142 base pairs) of GenBank accession AF138288 (SEQ ID. NO:1). The probe was 5′-6-FAM-TGAAGAACGCAGCGAAATGCGATAA-TAMRA, (SEQ ID. NO:4), where FAM and TAMRA were fluorescent dyes 6-carboxyfluorescein and 6-carboxy-N,N,N′,N′-tetramethylrhodamine, respectively. These sequences (total 58 nucleotides) match significantly to those of Fusarium (53 of 58, F. solani, AF178419 (SEQ ID. NO:5)) and Scedosporium (54 of 58, S. prolificans, AF022485 (SEQ ID. NO:6)) and allowed amplification of these moulds as well. The sequence homology to other rare moulds, however, was not assessed nor amplification tested.

Probes were designed to (1) achieve maximal energy transfer; (2) complete hybridization with the genomic DNA template; and (3) to be species-specific. The hybridization probes designed after extensive similarity analyses between Aspergillus species, other fungi, bacterial species, and human rRNA. The generic probe is conserved among Aspergillus species and contains one mismatch in the center among Fusarium species, several mismatches with Candidaand no mismatches with the human 5.8s RNA. Specific probes were designed to distinguish among Aspergillus species.

TABLE 3
Hybridization probes to detect species-specific Aspergillus isolates

| | | |
|---|---|---|
| Hybridization probes | Hybridization probes are located in | |
| Species-specific Aspergillus | the range of 507-53 5 in DNA | |
| probe | sequences | SEQ ID NO: |

| | | |
|---|---|---|
| A. niger | CTGCCGACGTTTTCCAACCAT | 14 |
| A. flavus | TTGCCGAACGCAAATCAATCT | 15 |
| A. fumigatus | CAGCCGACACCCAACTTTATT | 16 |
| A. terreus | CCGCCGACGCATTTATTTGCA | 17 |

The amplification primers were designed so that they are conserved among most Aspergillus species including fumigatus, nidulins, terrus, and favus. The forward primer contains one mismatch with Candidaspecies and the reverse primer contains several mismatches. There are no known significant matches with any human genome sequences in the databank using BLAST analysis. There are matches with dozens other species such as Alternaria, Ulocaldium, pennicillium, mycosphaerella, Platismatia. There are no similarity with bacterial species.

PCR™ Reagent Preparation.

PCR™ Master-mix was prepared as in table 4 below.

TABLE 4

| Components | Stock Concentration | Volume | Final Concentration | |
|---|---|---|---|---|
| Universal buffer A | 10× | 5 μl | | |
| MgCl₂ | 25 mM | 10 μl | 5 | mM |
| DNTP | 10 mM | 2 μl | 0.4 | mM |
| Forward primers | 50 pmol/μL | 1 μl | 1 | pmol |
| Reverse primers | 50 pmol/μL | 1 μl | 1 | pmol |
| TAMRA probe | 10 pmol/μL | 1 μl | 0.2 | pmol |
| H₂O (allow 10 μL | | 14.7 μL | | |
| for patient DNA and | | | | |
| 0.3 μL for enzyme) | | | | |

The guanidine thiocyanate (GuSCN) solution was prepared by dissolving 591 grams of GuSCN with 1 L water. The lysis buffer was prepared by adding 50 mM MOPS (pH 7.2/HCl), 0.2% Triton X-100, and 15% v/v) ethanol to a total volume of 1 L. The Wash Buffer, prepared by adding 50 mM Tris (pH 7.2/HCl) and 15% ethanol to a total volume of 1 L.

Preparation of Silica Suspension.

Silica beads were prepared before use to ensure uniformity of particle size and remove contaminants. Large quantity can be prepared at one time and stored indefinitely. The following procedure was used: 0.5 grams of silica were suspended in 1.5 mL epitube filled with water and autoclaved at 121° C. for 20 minutes. Thereafter 1.5 mL of 0.1 M sodium acetate (pH 5.2/HCl) was added and vortex to mix thoroughly, and spun in microfuge at 1000 rpm for 1 minute. This step was repeated at least 5 times. The silica was then spun at 14,000 rpm for 1 minute and the supernatant removed. 100 μL of 01. M sodium acetate (pH 5.2/HCl) was added and store at 4° C. until use. Tris-Acetate (TE) Buffer at pH 7.6 was prepared using 10 mM Tris-Cl and 1 mM EDTA;

Amplification.

Real-time PCR™ was performed on Prism 7700 Sequence Detection System (Applied Biosystems, Foster City, Calif.). Purified serum DNA (total up to 1 μg in 20 μl) was added to the reaction mix (final 25 μl) containing 1 μM each of the primers, 200 nM fluorescent-labeled probe, 400 μM dNTP, 5 mM MgCl₂, and 1.5U TaqGold polymerase. Following activation of polymerase (95° C.×10 min), 40 thermocycles were run with denaturation (95° C.×20 s), annealing (63° C.×20 s) and extension (72° C.×30 s). The last extension was 2 min. To prevent contamination, universal precautions and one-way flow of DNA extraction and amplification were exercised. To avoid potential subjectivity, the IMI status of each patient was unknown during the PCR™ assay.

Data Analysis.

Where appropriate, statistical analysis was performed by using either chi-square or the Fisher's exact test.

### Example 2

### Results

Specificity and Detection Range.

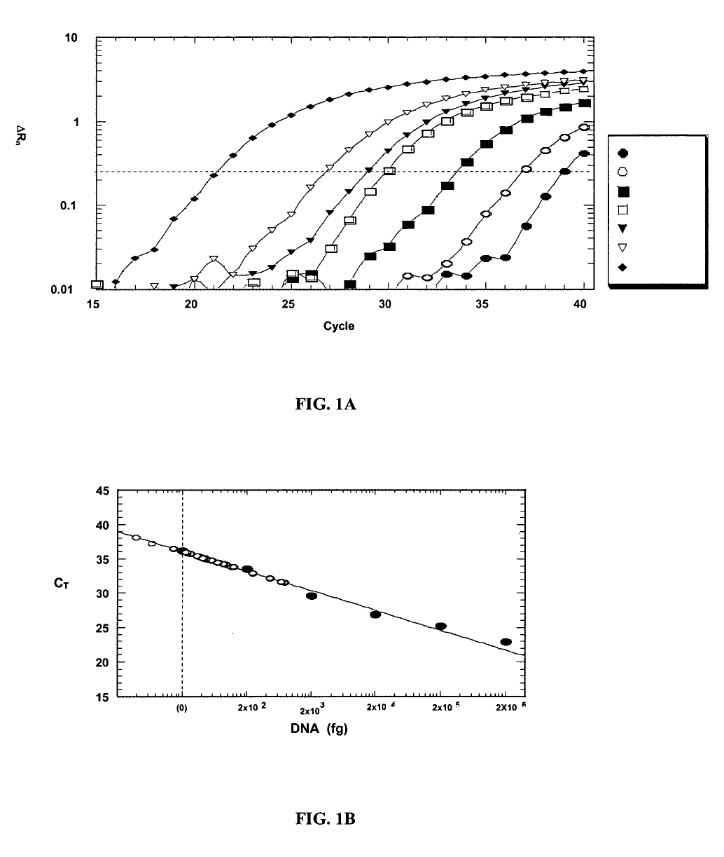

The specificity and detection range of the real-time PCR were assessed with purified Aspergillus, human, and candidal DNA. Neither human nor candidal DNA was amplified (data not shown). With normal human DNA as a background, purified Aspergillus DNA from 20 ng to 200 fg (5-log range) was detected at various amplification cycles (FIG. 1A). A logarithmic plot of the DNA quantity correlated linearly with the number of cycles (FIG. 1B), thus providing a basis for quantitative analysis of patient specimens.

Test of Sera.

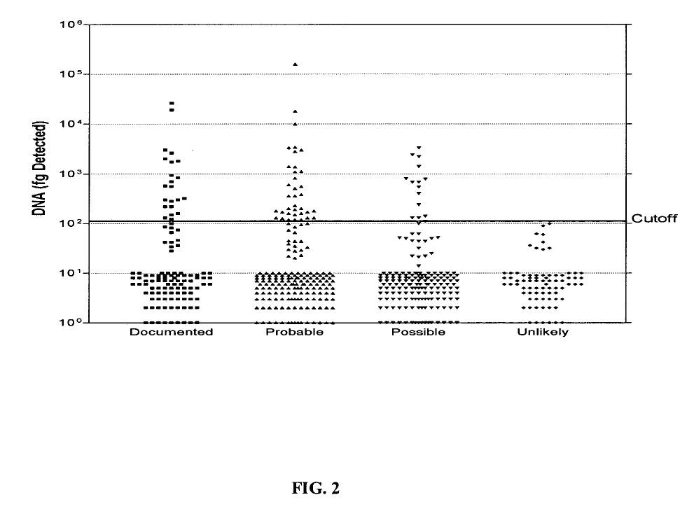

A total of 559 serum samples from 106 patients were tested with this real-time PCR assay and the results are shown in FIG. 2. All 76 sera from 35 patients with no evidence of IMI showed undetectable (less than negative control, <10 fg) or very low (10-100 fg) DNA levels so that a cutoff of 110 fg could be drawn confidently. This cutoff approximated 3 mould genomes for sensitivity and also enabled excellent specificity of the assay (100%). It allowed objective assessment of a positive test and categorization as weak (110-999 fg), moderate (1,000 to 10,000 fg), or strong (>10,000 fg) positivity (FIG. 2). At this cutoff, sera from patients with documented and suspected (probable and possible) IMI showed varying percentages of positivity that correlated with the diagnostic certainty of IMI, as shown in FIG. 2.

The test results are also summarized in Table 5. A patient was considered test-positive if one or more of his/her sera was positive. It was realized that this practical definition might cause potential bias towards more positive patients, due to multiple sera per patient, in the documented and suspected IMI groups as compared to fewer such serial sera in the unlikely IMI group. To minimize this, comparison was also made by the number of sera per group. Sera from documented and probable IMI had the same positive rate of 19%, which probably reflected similar disease status of the two groups. Many patients with probable IMI were short of tissue confirmation because of deeper lesions in the lung and risk factors unsuitable for invasive procedures. Possible IMI sera had a positive rate of 9% (15 of 176), significantly lower than 19% (58 of 307, combined documented and probable IMI) (X²=9.41, p<0.01). The overall positive rate for sera of these groups was 15% (73 of 483). When analyzed by the number of patients, the positive rate was 59% (42 of 71) for these three groups. A gradient was similarly seen with 40% (12 of 30 patients) for possible IMI, 68% (19 of 28) for probable IMI, and 85% (11 of 13) for documented IMI. Those patients in each group had mean 6 to 9 sera with median sampling time span of 10-16 days, suggesting good comparability.

TABLE 5
Serum real-time PCR^{TM} for patients with varying probability of IMI

| IMI | No. | No. | % | No. | No. | % | Median Sample |
|---|---|---|---|---|---|---|---|
| Category | Patients | Positive | Positive | Sera | Positive | Positive | span^{a}(day) |
| Documented | 13 | 11 | 85 | 120 | 23 | 19 | 13 |
| Probable | 28 | 19 | 68 | 187 | 35 | 19 | 36 |
| Possible | 30 | 12 | 40 | 176 | 15 | 9 | 10 |
| Subtotal | 71 | 42 | 59 | 483 | 73 | 15 | 16 |
| Unlikely | 35 | 0 | 0 | 76 | 0 | 0 | 1 |
| Total | 106 | 42 | 40 | 559 | 73 | 13 | 7 |

^{a}See Materials and Methods for detail.

An attempt was also made to analyze all the 73 positive sera and estimate the associated mould DNA burden in those patients (Table 6). At the assay cutoff of 110 fg, using dilution factor of 2.5 and total serum volume of 3,500 ml (70% of 5,000 ml blood volume), total body circulating mould DNA was estimated to be 3×10⁴ genomes. At the geometric mean positive level of 580 fg, the burden was 5.3 ng or 1.6×10⁵ genomes. The heaviest burden corresponded to 4.2×10⁷ genomes or 42 μg in a patient with end stage IA.

TABLE 6
Estimated Mould DNA Burden From Positive Sera (n = 73)

| | Geometric | Range |
|---|---|---|
| | mean | (n = 73) |
| DNA detected (fg) (copies of | 580 (17) | 110-160,000 (3-4,800) |
| genome) | | |
| Circulating DNA | 1,500 | 280-400,000 |
| concentration (fg/ml)^{a} | | |
| Total circulating DNA (ng) | 5.3 (160) | 1.0-1,400 (30-42,000) |
| (genomes × 10³)^{b} | | |

^{a}Dilution factor = 2.5
^{b}Estimated circulating serum volume of 3500 ml; each ng = ˜30,000 copies of mould (Aspergillus) genome; 1 ng = 10⁶ fg

Analysis of Mould Isolates.

From 1998 to 2000, 406 patients at the MD Anderson Cancer Center (Houston, Tex.) had Aspergillus isolated from the respiratory tract and/or tissue. Among these patients, the Aspergillus distributions were A. fumigatus in 97 patients (24% of 406), A. Flavus in 93 (23%), A. terreus in 87 (21%), A. niger in 75 (18%), other species in 10 (2%), and multiple species in 44 (11%). During the same period, Fusarium was isolated from 73 patients and Scedosporium from 13 patients. In this study, patients from the IMI groups represented a portion during this period and their PCR results were analyzed according to the mould types (Table 7). Consistent with the 3-year data and literature findings, A. fumigatus and A. flavus were the most common species and 52% of these patients (13 of 25) were shown to be positive by PCR™. Notably, A. terreus was isolated from 14 patients (9 as single isolate and 5 more as second species), 11 of whom had probable IMI and 3 possible IMI. Nine of the 14 patients (64%) were also PCR-positive. Therefore, A. terreus, being isolated nearly as common as A. fumigatus and A. Flavus, represented another major species associated with IA. In contrast, only 4 patients (3 possible and 1 probable IA) had A. niger isolated alone, and none was PCR-positive. This rate (0/4) was significantly lower than that of all other Aspergillus spp. combined (58%, 32 of 55) (p=0.039, Fisher's exact test). This finding is consistent with the literature that A. niger is infrequently associated with IA (Paterson and Singh, 1999; Latge, 1999).

Some of the IMI patients were afflicted by Fusarium, a mould that is difficult to be distinguished from Aspergillus in tissue phase by morphology. Fusarium can be cultured from the bloodstream and, unsurprisingly, all 6 patients with fusariosis showed detectable DNA in the serum (Table 7). One patient with probable scedosporiosis also tested positive (Table 7). Three of five patients who had possible IMI based on strong clinical suspicion without a mould being cultured also tested positive. This result is not surprising in view of previous studies by the inventors which demonstrated frequent culture failure for moulds (Tarrand et al., 2000).

TABLE 7
Mould isolates and serum PCR^{TM} from patients with
documented and suspected IMI

| Mould | No. of patients | PCR (+) | % |
|---|---|---|---|
| Aspergillus spp. | | | |
| A. fumigatus | 14 | 8 | 57 |
| A. flavus | 11 | 5 | 45 |
| A. terreus | 9 | 7 | 78 |
| A. niger | 4 | 0 | 0 |
| Other species | 10 | 7 | 70 |
| Two or more species | 11 | 5 | 45 |
| Subtotal | 59 | 32 | 54 |
| Fusarium | 6 | 6 | 100 |
| Scedosporium | 1 | 1 | 100 |
| Clinical diagnosis only^{a} | 5 | 3 | 60 |
| Total | 71 | 42 | 59 |

^{a}These five patients all had possible IMI, based on strong clinical suspicion.

Thus, the present invention provides real-time PCR-based methods for detecting septate mould DNA specifically, sensitively, and quantitatively in the sera of patients with IMI. The specificity was found to be excellent, being test-negative in all patients with no evidence of IMI. The sensitivity was also good, being 85% among patients with documented IMI that were diagnosed by a combined assessment of histology, microbiology, radiology and clinical manifestations. This assay is convenient and minimally invasive, thus making it a potential alternative to other invasive diagnostic procedures, such as biopsy and open surgery. In many patients with hematological malignancies, these procedures are too risky to be performed due to bleeding, additional infections, etc. Thus, this assay eliminates the possibility of cross-contamination by PCR™ products since simultaneous probing and detection omits the post-PCR manipulation that is needed in conventional PCR™ assays and prone to contamination. Like any other diagnostic tests, multiple sampling may be necessary. Since most positive patients had IA (at least 76%, 32 of 42 patients, Table 7), a positive test may be presumptive of IA, until the identification of the mould by culture.

The outcome of IMI and the level of circulating mould DNA may also be influenced by several factors, such as the use of anti-fungal therapeutics, remission of underlying disease, and resolution or improvement of active IMI or its risk factors. Many of the patients in this group were also treated with experimental anti-flugals in addition to amphotericin and itraconazole.

A. terreus was also found to be a frequent cause of IA in this study in addition to A. fumigatus and A. flavus. Patients with suspected A. terreus infection alone showed the highest positive rate for circulating DNA (7 of 9, Table 7), raising the possibility that fungemia may be more common with this species. Indeed, the inventors previous study also reported true fungemia caused by A. terreus, but not by other Aspergillus species (Kontoyiannis et al., 2000). Thus, it is speculated that A. terreus may be more angio-invasive and further study is needed to gain insight into this aspect. The overall recovery of Aspergillus from blood culture is very low, which precludes assessment of circulatory burden imposed by this organism. Possible explanations include fragility or low numbers of viable organisms in the bloodstream, transient shedding into the circulation, inhibitory factors in the blood, failure of current culture techniques, or combinations of these. Nonetheless, the present quantitative PCR™ allowed estimation of the mould DNA in the circulation (free, cell-bound, and fragmented forms from viable or dead organisms). At the lowest detectable level, circulative mould burden was ˜3×10⁴ genomes. The geometric mean was 5-fold higher (1.6×10⁵ genomes) and the heaviest burden was 4.2×10⁷ genomes (Table 6). In a recent mouse model study of disseminated aspergillosis, quantitative PCR™ has also been used to measure the burden of A. fumigatus, which correlated with disease progression and therapeutic efficacy (Bowman et al., 2001). The inventors are currently investigating these issues in patients.

Galactomannan is a complex polysaccharide antigen and major cell wall constituent of Aspergillus (Latge, 1999). Detection of galactomannan in the circulation by immunoassay has also become a useful diagnostic tool for IA. A recent prospective study revealed a sensitivity of 90% and a specificity of 98% (Maertens et al., 2001). These results are similar to the findings by real-time PCR™. However, false positive reactions are relatively frequent in galactomannan assays, particularly in the format of enzyme-linked immunosorbent assay, being 14% in the above-mentioned study and 8% in another (Stynen et al., 1995). Admittedly, lack of specificity can also be problematic with PCR™ due to the exponential amplification of the target DNA. For instance, non-specificity was a likely factor in a nested PCR™ study that showed unusually high positive rates of sera, 11% for patients with unlikely and indeterminate IA and 61% for patients with possible to proven IA (Williamson et al., 2000). In the present invention, this potential pitfall was avoided or minimized through careful design of primers and probe and selection of cutoff value. As such, the overall serum positive rate of 15% in the IMI groups (Table 2) is more realistic. The present methods based on real-time PCR™, being more specific and comparably sensitive, would correct the false positive results by the galactomannan assay if both assays were used in suspected IA patients.

Efficient DNA extraction is crucial for any blood-based PCR™ assay, particularly to detect microbial DNA. The silica-binding method used in this study was able to extract extremely low levels (picogram) of fungal DNA along with microgram levels (10⁶-fold more) of human DNA in a serum sample. It was superior to the phenol-chloroform method and some commercial DNA extraction kits (data not shown). A previous study (Fahle, et al., 2000), also reported that the silica-binding method was the best among six methods in extracting cytomegalovirus DNA from serum and cerebrospinal fluid. The inventors chose serum to extract mould DNA because it appears to be a better source than plasma or whole blood for the extraction of fungal or bacterial DNA (Yamakami et al., 1996; Zerva et al., 2001). In addition, serum typically has much less human DNA (1-5 μg/ml) than whole blood (30-50 μg/ml), which might favor the extraction of minute quantities of exogenous microbial DNA.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.
U.S. Pat. No. 4,659,774
U.S. Pat. No. 4,683,202
U.S. Pat. No. 4,800,159
U.S. Pat. No. 4,816,571
U.S. Pat. No. 4,959,463
U.S. Pat. No. 5,141,813
U.S. Pat. No. 5,264,566
U.S. Pat. No. 5,428,148
U.S. Pat. No. 5,547,861
U.S. Pat. No. 5,554,744
U.S. Pat. No. 5,574,146
U.S. Pat. No. 5,602,244
U.S. Pat. No. 5,843,650
U.S. Pat. No. 5,843,663
U.S. Pat. No. 5,846,709
U.S. Pat. No. 5,846,783
U.S. Pat. No. 5,849,481
U.S. Pat. No. 5,849,486
U.S. Pat. No. 5,849,497
U.S. Pat. No. 5,849,546
U.S. Pat. No. 5,849,547
U.S. Pat. No. 5,851,772
U.S. Pat. No. 5,858,652
U.S. Pat. No. 5,866,366
U.S. Pat. No. 5,900,481
U.S. Pat. No. 5,912,148
U.S. Pat. No. 5,916,776
U.S. Pat. No. 5,916,779
U.S. Pat. No. 5,919,626
U.S. Pat. No. 5,922,574
U.S. Pat. No. 5,928,905
U.S. Pat. No. 5,928,906
U.S. Pat. No. 5,932,451
U.S. Pat. No. 5,935,825
U.S. Pat. No. 5,939,291
U.S. Pat. No. 5,942,391
Anaissie, Clin. Infect. Dis., 17:1022-1031, 1993.
Ascioglu et al., Clin. Infect. Dis., 34:7-14, 2000.
Ascioglu et al, J. Clin. Dis., 34:7-14, 2002.
Baddley et al, Clin. Infect. Dis., 32(9):1319-24, 2001.
Binninger et al., Diagn Mol Pathol., Dec;1(4):239-45, 1992.
Bodey and Vartivarian, Eur. J. Clin. Microbiol. Infect., 8(5):413-437, 1989.
Boom et al., J. Clin. Microbiol., 28:495-503, 1990.
Bowman et al, Antimicrob. Agents Chemother., 45:3474-81, 2001.
Brakhage and Langfelder, Annu. Rev. Microbiol., 56:433-455, 2000.
Bretagne et al., Clin. Infect. Dis., 26:1407-1412, 1998.
Bumes, J Antimicrob. Chemother., 28:23-33, 1991.
Chomczynski and Sacchi,Anal Biochem., Apr; 162(1):156-9, 1987.
Choudhury et al., (Indian) J. Pharmaceut. Sci., 63(1):58-60, 2001.
de Repentigny, Clin. Infect. Dis., 14: S11-S22, 1992.
Denning, Rev. Infect. Dis., 12:1147-1201, 1990.
Einsele et aL., J Clin. Microbiol., 35:1353-60, 1997.
Erjavec and Verweij, Drug Resistance Updates, 5:3-10, 2002.
European Application No. 320308
European Application No. 329 822
Fahle and Fischer, J. Clin. Microbiol., 38:3860-3863, 2000.
Fahle et al., J. Clin. Microbiol., 38:3860-3, 2000.
Forster Ann. Phys. 2:55-75, 1948.
Frohman, In: PCR Protocols: A Guide To Methods And Applications, Academic Press, N.Y., 1990.
GB Application No. 2 202 328
Goodman, N. Engl. J. Med., 326:845-851, 1992.
Greaves, In: Histopathology of preclinical toxicity studies: Interpretation and Relevance in drug safety evaluation, 864-865, 2000.
Haynes et al., J. Clin. Microbiol., 28:2040-2044, 1990.
Hebart et al., J Infect. Dis., 181:1713-9, 2000.
Higuchi et al., Biotechnology, 10:413-417 1992.
Higuchi et al., Biotechnology, 10:413-417, 1992.
Higuchi et al., Biotechnology, 11(9):1026-30, 1993.
Holland, Proc. Nat. Acad. Sci. USA, 88:7276-7280, 1991.
Innis et al., PCR Protocols, Academic Press, Inc., San Diego Calif., 1990.
Itakura and Riggs, Science, 209:1401-1405, 1980.
Kontoyiannis et al., Clin. Infect. Dis., 31:188-189, 2000.
Koster et al., In: Biomedical Environmental Mass Spectrometry, 14:111-116, 1987.
Kwoh et al., Proc. Nat. Acad. Sci. USA, 86: 1173, 1989.
Latge Clin. Microbiol. Rev., 12:310-50, 1999.
Latge, In: Tools and trends on the detection of aspergillus fumigatus, Current Topics in Medical Mycology, Springer-Verlag, NY, 1985.
Lee et al., Nuc. Acids Res., 21:3761-3766, 1993.
Lee, Nucleic Acids Res., 21:3761-3766, 1993.
Maertens et al., Blood, 97:1604-10, 2001.
Maniatis et al., Molecular Cloning, A Laboratory Manual, Cold Spring Harbor Press, Cold Spring Harbor, N.Y., 1988.
Martins et al., J. Clin. Microbiol., 33:940-943, 1995.
Ohara et al., Proc. Natl. Acad. Sci. USA, 86:5673-5677, 1989.
Oliveria et al., Braz. J. Med. Biol. Res., 35(7):789-98, 200, 2002.
Paterson and Singh, Medicine, 78:123-128, 1999.
PCT Appln. PCT/US87/00880
PCT Appln. PCT/US89/01025
PCT Appln. WO 88/10315
PCT Appln. WO 89/06700
PCT Appln. WO 90/07641
PCT Appln. WO 96/21144
Pryce et al., Diag. Microbiol Infect. Dis., 47:487-496, 2003.
Sambrook, Molecular Cloning: A Laboratory Manual, 3rd Ed., Cold Spring Harbor Press, Cold Spring Harbor, N.Y., 2001.
Soubani et al., Chest, 121(6):1988-1999, 2002.
Stynen et al., J. Clin. Microbiol., 33:497-500, 1995.
Tang, J Clin. Pathol., 45:1-5, 1992.
Tarrand et al., 38^{th} Annual Meeting of the Infectious Diseases Society of America (IDSA), abstract #276, 2000.
Treger, J. Infect. Dis., 152:572-576, 1985.
Van Burik et al., J. Clin. Microbiol., 36:1169-75, 1998.
Vogeser et al., Eur. J. Clin. Microbiol. Infect. Dis., 16:1-6, 1997.
Walker et al., Proc. Natl. Acad. Sci. USA, 89:392-396 1992.
Williamson et al., Br. J. Haematol., 108:132-9, 2000.
Yamakami et al., J. Clin. Micro., 34(10), 1996.
Yamakami et al., J. Clin. Microbiol., 34:2464-2468, 1996.
Zerva et al., J. Clin. Microbiol., 39:1661-4, 2001.

1. A method for detecting the presence of a mould infection in a subject comprising identifying 5.8S ribosomal RNA of a mould, or a DNA encoding said RNA in a sample obtained from the subject.

2. The method of claim 1, wherein the mould is an invasive mould infection.

3. The method of claim 1, wherein the mould is a non-invasive mould infection.

4. The method of claim 1, comprising mixing the sample with a nucleic acid encoding said RNA.

5. The method of claim 4, comprising mixing the sample with a primer that hybridizes to the nucleic acid encoding said RNA.

6. The method of claim 4, further comprising amplifying the sample encoding said RNA.

7. The method of claim 6, comprising determining the presence or absence of an amplification product in the sample.

8. The method of claim 1, wherein the sample is a nucleic acid containing sample.

9. The method of claim 8, wherein the nucleic acid containing sample is a DNA sample.

10. The method of claim 8, wherein the nucleic acid containing sample is a RNA sample.

11. The method of claim 7, further comprising quantitating the amplification product whereby the amount of mould nucleic acid is quantitated.

12. The method of claim 11, wherein said quantitating comprises:
a) mixing a first probe capable of hybridizing to a nucleic acid sequence of said mould in an amplification reaction;
b) mixing a second probe capable of hybridizing to a standard nucleic acid that is amplified to a pre-determined quantity in the amplification reaction of step (a); and
c) comparing the signal of the amplification reaction of step (a) to the signal of the amplification reaction containing the standard nucleic acid.

13. The method of claim 12, wherein the comparing is in the exponential phase of the amplification.

14. The method of claim 12, wherein the first probe comprises nucleic acids that hybridize to the nucleic acid sequence of SEQ ID NO:1, SEQ ID NO:5 or SEQ ID NO:6 or fragments thereof.

15. The method of claim 12, wherein the second probe comprises nucleic acids that hybridize to the nucleic acid sequence of SEQ ID NO:1, SEQ ID NO:5 or SEQ ID NO:6 or fragments thereof.

16. The method of claim 12, wherein the probe comprises the sequence 5′-TGAAGAACGCAGCGAAATGCGATAA-3′ (SEQ ID NO:4).

17. The method of claim 12, wherein the probe comprises the sequence of SEQ ID NO:14, SEQ ID NO:15, SEQ ID NO:16, SEQ ID NO:17, SEQ ID. NO:18, SEQ ID NO:19, SEQ ID NO:20, or SEQ ID NO:21.

18. The method of claim 12, wherein said first or said second probe is labeled.

19. The method of claim 18, wherein the label is a fluorescent label.

20. The method of claim 19, wherein said fluorescent label is 6-carboxyfluorescein (FAM), 6-carboxy-N,N,N′,N′-tetramethylrhodamine (TAMRA), Alexa 350, Alexa 430, AMCA, BODIPY 630/650, BODIPY 650/665, BODIPY-FL, BODIPY-R6G,
BODIPY-TMR, BODIPY-TRX, Cascade Blue, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Texas Red, VIC, or DABCYL.

21. The method of claim 20, wherein the probe comprises the sequence 5′-6-FAM-TGAAGAACGCAGCGAAATGCGATAA-TAMRA-3′ (SEQ ID NO:4).

22. The method of claim 6, wherein the amplifying is preceded by a reverse transcription reaction.

23. The method of claim 1, wherein the mould belongs to Aspergillus species, Fusarium species, or Scedosporium species.

24. The method of claim 23, wherein said mould is of the Aspergillus species.

25. The method of claim 24, wherein said mould is Aspergillus fumigatus, Aspergillus flavus, Aspergillus terreus, Aspergillus vesicularis, Aspergillus nidulans, or Aspergillus niger.

26. The method of claim 23, wherein said mould is of the Fusarium species.

27. The method of claim 26, wherein said mould is Fusarium solani.

28. The method of claim 23, wherein said mould is of the Scedosporium species.

29. The method of claim 28, wherein said invasive mould is Scedosporium prolificans.

30. The method of claim 1, where said sample comprises serum, blood, plasma, cells, tissues, aspirates, biopsies, fine needle aspirates, skin biopsies, lymph fluid or urine.

31. The method of claim 1, where said sample comprises serum.

32. The method of claim 5, wherein said primers are comprised of nucleic acids that hybridize to the nucleic acid sequence comprised in SEQ ID NO: 1 or fragments or variant thereof.

33. The method of claim 32, wherein said primers comprise the nucleic acid sequence

| | |
|---|---|
| TTGGTTCCGGCATCGA. | (SEQ ID NO:2) |

34. The method of claim 32, wherein said primers comprise the nucleic acid sequence SEQ ID NO:9, SEQ ID NO:10, SEQ ID NO:13, SEQ ID NO:22, SEQ ID NO:23, SEQ ID NO:24, or SEQ ID NO:25.

35. The method of claim 32, wherein said primers comprise the nucleic acid sequence

| | |
|---|---|
| GCAGCAATGACGCTCGG. | (SEQ ID NO:3) |

36. The method of claim 32, wherein said primers comprise the nucleic acid sequence SEQ ID NO:11, SEQ ID NO:12, SEQ ID NO:26, SEQ ID NO:27, SEQ ID NO:28, or SEQ ID NO:29.

37. The method of claim 4, wherein said primers are comprised of nucleic acids that hybridize to the nucleic acid sequence comprised in SEQ ID NO: 5 or fragments or variant thereof.

38. The method of claim 4, wherein said primers are comprised of nucleic acids that hybridize to the nucleic acid sequence comprised in SEQ ID NO: 6 or fragments variant thereof.

39. The method of claim 4, wherein said primers are comprised of nucleic acids that hybridize to the nucleic acid sequence comprised in SEQ ID NO: 7 or fragments variant thereof.

40. The method of claim 4, wherein said primers are comprised of nucleic acids that hybridize to the nucleic acid sequence comprised in SEQ ID NO: 8 or fragments variant thereof.

41. The method of claim 6, wherein said amplifying is by polymerase chain reaction.

42. The method of claim 7, wherein said determining is in real time.

43. The method of claim 1, wherein the detecting is in a detection range of 1 fg to 20 ng of DNA.

44. The method of claim 1, wherein the detecting is in a detection range of 1 fg to 800 fg of DNA.

45. The method of claim 1, wherein the detecting is in a detection range of 100 fg to 200 fg of DNA.

46. The method of claim 1, further comprising obtaining said sample from the subject.

47. The method of claim 1, further comprising isolating nucleic acids from said sample.

48. A kit for detecting an mould in a biological sample comprising:
a) primers that hybridize to 5.8S ribosomal RNA of an mould, or DNA encoding said RNA; and
b) reagents for an amplification reaction comprising a heat-stable DNA polymerase enzyme, buffers, water, magnesium chloride, and deoxynucleotides;
each enclosed in a suitable container means.

49. The kit of claim 48, wherein the primers comprise nucleic acids to the nucleic acid sequence of SEQ ID NO:1, SEQ ID NO:5 or SEQ ID NO:6 or fragments thereof.

50. The kit of claim 48, wherein the primers comprise nucleic acids to the nucleic acid sequence of SEQ ID NO:7, or SEQ ID NO:8 or fragments thereof.

51. The kit of claim 48, wherein the primers comprise the nucleic acid sequence

| | |
|---|---|
| TTGGTTCCGGCATCGA. | (SEQ ID NO:2) |

52. The kit of claim 48, wherein the primers comprise the nucleic acid sequence SEQ ID NO:9, SEQ ID NO:10, SEQ ID NO:13, SEQ ID. NO:22, SEQ ID NO:23, SEQ ID NO:24, or SEQ ID NO:25.

53. The kit of claim 48, wherein the primers comprise the nucleic acid sequence

| | |
|---|---|
| GCAGCAATGACGCTCGG. | (SEQ ID NO:3) |

54. The kit of claim 48, wherein the primers comprise the nucleic acid sequence SEQ ID NO: 11, SEQ ID NO:12, SEQ ID NO:26, SEQ ID NO:27, SEQ ID NO:28, or SEQ ID NO:29.

55. The kit of claim 48, further comprising one or more probe that hybridize to ribosomal RNA of the mould or fragments thereof.

56. The kit of claim 55, wherein the ribosomal RNA comprises one or more probe that hybridize to the 5.8S ribosomal RNA of the mould or fragments thereof.

57. The kit of claim 55, wherein said probes comprise nucleic acids that hybridize to the nucleic acid sequence of SEQ ID NO:1, SEQ ID NO:5 or SEQ ID NO:6 or fragments thereof.

58. The kit of claim 55, wherein the probe comprises the sequence 5′-TGAAGAACGCAGCGAAATGCGATAA-3′ (SEQ ID NO:4).

59. The kit of claim 55, wherein the probe comprises the sequence of SEQ ID NO: 14, SEQ ID NO:15, SEQ ID NO:16, SEQ ID NO:17, SEQ ID NO:18, SEQ ID NO:19, SEQ ID NO:20, or SEQ ID NO:21.

60. The kit of claim 55, wherein the one or more probe is labeled.

61. The kit of claim 48, further comprising reagents to isolate nucleic acids from the sample.

62. The kit of claim 61, wherein said nucleic acid isolated is mRNA.

63. The kit of claim 61, wherein said nucleic acid isolated is DNA.

64. A method for purification of a nucleic acid encoding 5.8S ribosomal RNA of a mould in a nucleic acid containing sample comprising:
a) obtaining said nucleic acid containing sample from a subject;
b) incubating the sample with a lysis reagent for about at least 60 minutes;
c) vortexing the sample intermittently to mix; and
d) centrifuging the sample at greater than 3000×g for 5 min.

65. The method of claim 64, wherein the sample is incubated at about 50° C.

66. The method of claim 64, wherein the sample is incubated at about 37° C.

67. The method of claim 64, wherein the sample is centrifuged at about 6000×g for 15 minutes.

68. A method for enhancing binding of a nucleic acid encoding 5.8S ribosomal RNA of a mould to silica beads comprising:
a) washing the silica beads with sodium acetate of about pH 5.2;
b) mixing the silica beads of step (a) by vortexing; and
c) centrifuging the silica beads at about 1000 rpm for at least 1 minute.

69. The method of claim 68, wherein the silica beads of step (a) are washed at least 5 times.

70. The method of claim 69, wherein the silica beads are centrifuged at least at 12000 rpm.

71. The method of claim 69, comprising selecting silica beads of particle size ranging from 5 μM to 10 μM.

72. The method of claim 68, wherein the sodium acetate is about 0.05 M to 2.5 M.

73. The method of claim 72, wherein the sodium acetate is 0.1 M.

74. The method of claim 68, further comprising mixing said silica beads with a nucleic acid containing sample from a subject comprising a nucleic acid encoding 5.8S ribosomal RNA of a mould.
